(19) United States
(12) Patent Application Publication
(10) Pub. No.: US 2023/0001005 A1
(43) Pub. Date: Jan. 5, 2023

(54) TREATMENT OF CANCERS WITH ANTIBODY DRUG CONJUGATES (ADC) THAT BIND TO 191P4D12 PROTEINS

(71) Applicants: AGENSYS, INC., Northbrook, IL (US); SEAGEN INC., Bothell, WA (US)

(72) Inventor: Oyewale O. ABIDOYE, Bellevue, WA (US)

(73) Assignees: AGENSYS, INC., Northbrook, IL (US); SEAGEN INC., Bothell, WA (US)

(21) Appl. No.: 17/634,026

(22) PCT Filed: Aug. 10, 2020
(86) PCT No.: PCT/US2020/045567

§ 371 Date: Feb. 9, 2022

Related U.S. Application Data
(60) Provisional application No. 62/886,270, filed on Aug. 13, 2019.

(51) Int. Cl. A61K 47/68; A61P 35/00; A61P 11/00; A61K 38/07

(52) U.S. Cl. CPC A61K 47/6849; A61P 35/00; A61P 11/00; A61K 38/07; A61K 47/6889; C07K 2317/565; C07K 2317/55; C07K 2317/622; C07K 2317/21

(57) ABSTRACT

Provided herein are methods for the treatment of cancers with antibody drug conjugates (ADC) that bind to 191P4D12 proteins.

### CROSS-REFERENCE TO RELATED APPLICATIONS

This application is a U.S. National Stage Application under 35 U.S.C. § 371 of International Patent Application No. PCT/US2020/045567, filed Aug. 10, 2020, which claims the benefit of U.S. Provisional Patent Application No. 62/886,270, filed Aug. 13, 2019, the disclosure of each of which is incorporated by reference herein in its entirety.

### 1. FIELD

Provided herein are methods for treating cancers with antibody drug conjugates (ADC) that bind to 191P4D12 proteins.

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. https://seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. https://www.who.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a type I transmembrane protein and member of a family of related immunoglobulin-like adhesion molecules implicated in cell-to-cell adhesion. 191P4D12 belongs to the Nectin family of adhesion molecules. 191P4D12 is composed of an extracellular domain (ECD) containing 3 Ig-like subdomains, a transmembrane helix, and an intracellular region (Takai Y et al, Annu Rev Cell Dev Biol 2008; 24:309-42). Nectins are thought to mediate Ca2+-independent cell-cell adhesion via both homophilic and heterophilic trans interactions at adherens junctions where they can recruit cadherins and modulate cytoskeletal rearrangements (Rikitake & Takai, Cell Mol Life Sci. 2008; 65(2):253-63). Sequence identity of 191P4D12 to other Nectin family members is low and ranges between 25% to 30% in the ECD (Reymond N et al, J Biol Chem 2001;43205-15). Nectin-facilitated adhesion supports several biological processes, such as immune modulation, host-pathogen interaction, and immune evasion (Sakisaka T et al, Current Opinion in Cell Biology 2007; 19:593-602).

Breast Cancer

Globally, there will be approximately 2.1 million newly diagnosed female breast cancer cases in 2018, accounting for almost 1 in 4 cancer cases among women. The disease is the most frequently diagnosed cancer in the vast majority of countries and is also the leading cause of cancer-related death in women. Following metastatic diagnosis, prognosis is poor with a with a 5-year survival rate of approximately 15%.

The selection of appropriate therapy for metastatic breast cancer is complex because of the many treatment options and biologic heterogeneity of the disease. The potential treatment options are influenced by estrogen and progesterone receptors and by human epidermal growth factor receptor 2 (HER2) status of the tumor. Treatment options for subjects presenting with metastatic breast cancer may also be influenced by what adjuvant therapy was used, how soon after adjuvant therapy the subject relapses, and by sites of metastasis.

Hormone Receptor Positive, Human Epidermal Growth Factor Receptor 2 Negative Breast Cancer

Hormone receptor positive (HR+)/HER2−negative breast cancer is the most common breast cancer subtype (>70%), occurring predominantly in postmenopausal women. The initial treatment for women with metastatic disease consists primarily of endocrine therapy. This is usually administered alone, in combination with a CDK4/6 inhibitor, or as dual endocrine blockade. For women who are endocrine refractory or women who have symptomatic visceral disease, systemic chemotherapy is recommended.

Several cytotoxic chemotherapy agents have shown activity in metastatic breast cancer, including anthracyclines, taxanes, gemcitabine, capecitabine, vinorelbine, eribulin and ixabepilone. The response rates with these agents vary depending on the type of prior therapy, as well as the breast cancer subtype. In general, anthracycline-based combination therapy and taxanes such as paclitaxel and docetaxel are thought to be the most active (Piccart M, Clin Breast Cancer 2008;100-13). Given the wide use of anthracyclines in the adjuvant setting and the increased risk of cardiotoxicity, the use of anthracyclines in the metastatic setting is limited. Taxanes are the most commonly used agent for patients with locally advanced or metastatic disease, particularly in the front-line setting (Greene & Hennessy, J Oncol Pharm Pract 2015;201-12). Sequential single agent therapies are recommended over combinations due to lower toxicities and limited survival benefit. Responses to commonly used single agent chemotherapy patients with HR+/HER2−negative breast cancer are primarily limited to subgroup analysis, these have ranged between 11% to 36% (Robson M et al, N Engl J Med. 2017; 377(18):1792-3; Kaufman PA et al, J Clin Onco. 2015; 33(6):594-601; Cortes Jet al, Lancet. 2011; 377:914-23). In general, responses tended to be lower in pretreated patients with reported ranges between 10% to 13% (Perez E A et al, J Clin Oncol. 2007; 25:3407-14; Jones S et al, J Clin Oncol. 1995; 13(10):2567-74).

Triple Negative Breast Cancer

Triple negative breast cancer (TNBC) is defined by the absence of immunostaining for estrogen receptor (ER), progesterone receptor (PR) and HER2. Overall, approximately 15% to 20% of breast cancers are classified as TNBC. TNBC is associated with aggressive tumor biology, visceral metastasis, and a poor prognosis (Plasilova ML et al, Medicine (Baltimore). 2016;95(35):e4614).

Taxane-based regimens are considered a standard of care in first-line therapy for patients with metastatic breast cancer, including TNBC. More recently the FDA granted accelerated approval for atezolizumab in combination with nab-paclitaxel for the treatment of patients with unresectable locally advanced or metastatic TNBC whose tumors express programmed death-ligand 1 (PD-L1; median progression-free survival [PFS] of 7.5 months versus 5.0 months; objective response rate (ORR) of 56% versus 46%) (Schmid P et al, N Engl J Med. 2019; 380(10):987-988). No standard approach exists for second- or further-line treatment, and options for chemotherapy are the same as those for other subtypes. Single-agent cytotoxic chemotherapeutic agents are generally preferred over combination chemotherapy due to the lack of survival benefit and increased toxicity except in the setting of aggressive disease and visceral involvement (Cardoso F et al, Ann Oncol. 2017; 28(2):208-217; National Comprehensive Cancer Network, 2017, Non-small cell lung cancer, NCCN clinical practice guidelines in oncology (NCCN guidelines), http://www.nccn.org/professionals/physician_gls/pdf/nscl.pdf. Accessed 5 Jun. 2019). Standard chemotherapy among pretreated patients is associated with low response rates (10% to 15%) and short progression-free survival (2 to 3 months) (Hurvitz & Mead, Curr Opin Obstet Gynecol. 2016; 28(1):59-69).

Non-Small Cell Lung Cancer

Lung cancer (both small cell and non-small cell) is the leading cause of cancer deaths in the US (American Cancer Society. Key Statistics for Lung Cancer. 8 Jan. 2019a. https://www.cancer. org/cancer/non-small-cell-lung-cancer/about/key-statistics. html. Accessed 5 Jun. 2019]. Most patients diagnosed with lung cancer are 65 years of age or older and, the average age at the time of diagnosis is approximately 70 years of age.

Non-small cell lung cancer (NSCLC) accounts for approximately 85% of all lung cancers (Tan & Huq, Non-Small Cell Lung Cancer (NSCLC), Apr. 13, 2019, https://emedicine.medscape.com/article/279960-overview, accessed 5 Jun. 2019; American Cancer Society: What is non-small cell lung cancer, 16 May 2016, https://www.cancer.org/cancer/non-small-cell-lung-cancer/about/what-is-non-small-cell-lung-cancer.html, accessed 5 Jun. 2019) and can be subclassified as squamous (approximately 30% of NSCLC cases) and non-squamous (approximately 40% of NSCLC cases) histological types (American Cancer Society. Non-Small Cell Lung Cancer. 2019b. http://www.cancer.org/Cancer/LungCancer-Non-SmallCell/DetailedGuide/lung-cancer--non-small-cell--non-small-cell-lung-cancer. Accessed 5 Jun. 2019).

Squamous Non-Small Cell Lung Cancer

Squamous NSCLC is a distinct histological subtype of NSCLC that is challenging to treat as a result of specific patient and disease characteristics, which include older age, metastatic (including malignant or metastatic malignant) disease at diagnosis, comorbid disease, and the central location of tumors (Socinski M et al, Cell Lung Cancer 2018;165-183). These characteristics have a bearing on treatment outcomes in metastatic (including malignant or metastatic malignant) squamous NSCLC, resulting in a median survival rate of approximately 30% shorter than for patients with other NSCLC subtypes.

There are limited treatment options, especially for first-line treatment of metastatic (including malignant or metastatic malignant) squamous NSCLC, with a resultant impact on survival outcomes (National Comprehensive Cancer Network. 2017, Non-small cell lung cancer, NCCN clinical practice guidelines in oncology (NCCN guidelines), http://www.nccn.org/professionals/physician_gls/pdf/nscl.pdf, accessed 5 Jun. 2019; Novello S et al, Ann Oncol 2016;27 (Supple 5):v1-v27; Masters GA et al, J Clin Oncol 2015; 33(30):3488-3515). Given the recent approvals of targeted therapies and immunotherapies for metastatic (including malignant or metastatic malignant) NSCLC and continuation towards personalization of lung cancer treatment, there is also a need to evaluate the effectiveness of these new treatments for metastatic (including malignant or metastatic malignant) squamous NSCLC.

Non-Squamous Non-Small Cell Lung Cancer

Non-squamous NSCLC is a heterogeneous disease with multiple treatment options dependent upon staging, presence of metastasis, and patient factors, including presence of comorbidities among other considerations. As such, current treatment options include surgical resection, chemotherapy, radiation, immunotherapy, and targeted therapy. Currently, the first-line therapy for patients with metastatic (including malignant or metastatic malignant) non-squamous NSCLC without targetable genetic aberrations is platinum-doublet chemotherapy. With the exception of bevacizumab, and despite extensive study of multiple targeted and cytotoxic agents, the addition of a third agent to platinum-doublet chemotherapy has not been shown to improve progression-free or OS over platinum-doublet chemotherapy alone in randomized studies (Reck M et al, Ann Oncol 2010; 1804-09; Sandler A et al, N Engl J Med 2006; 355:2542-50).

Head and Neck Cancer

Head and neck cancer is a group of cancers that starts in the mouth, nose, throat, larynx, sinuses, or salivary glands (National Cancer Institute, Head and Neck Cancers, 29 Mar. 2017, https://www.cancer.gov/types/head-and-neck/head-neck-fact-sheet, accessed 5 Jun. 2019). Worldwide, head and neck cancers have affected more than 5.5 million people (mouth 2.4 million, throat 1.7 million, and larynx 1.4 million) and caused over 379000 deaths (GBD. 2016a. Global, regional, and national incidence, prevalence, and years lived with disability for 310 diseases and injuries, 1990-2015: a systematic analysis for the Global Burden of Disease Study 2015, accessed 5 Jun. 2019, https://www.thelancet.com/journals/lancet/article/PIIS0140-6736(16)31678-6/fulltext; GBD. 2016b. Global, regional, and national life expectancy, all-cause mortality, and cause-specific mortality for 249 causes of death, 1980-2015: a systematic analysis for the Global Burden of Disease Study 2015, https://www.sciencedirect.com/science/article/pii/S0140673616310121, accessed 5 Jun. 2019). Worldwide, approximately 600000 cases of head and neck cancers will arise this year, and only 40% to 60% of patients will survive for 5 years (Rene Leemans C, et al. The molecular biology of head and neck cancer, Nature Reviews Cancer, 16 Dec. 2011, accessed 5 Jun. 2019, https://www.nature.com/articles/nrc2982).

The most important risk factors are tobacco use and alcohol consumption, which seem to have a synergistic effect (Decker & Goldstein, N Engl J Med. 1982;1151-1155). A subgroup of head and neck cancers, particularly those of the oropharynx, are caused by infection with high-risk types of human papillomavirus (HPV) (Rene Leemans C, et al. The molecular biology of head and neck cancer, Nature Reviews Cancer, 16 Dec. 2011, accessed 5 Jun. 2019, https://www.nature.com/articles/nrc2982).

Treatment is largely determined by the stage at presentation, but may include a combination of surgery, radiation therapy, chemotherapy, and targeted therapy (National Cancer Institute, 2019, Cancer Stat Facts: Cancer of Any Site, https://seer.cancer.gov/statfacts/html/all.html, accessed 5 Jun. 2019). Survival, however, has not markedly improved in recent decades because patients often develop locoregional recurrences, distant metastases and second primary tumors. The limited information available on the molecular carcinogenesis of head and neck cancers, and the genetic and biological heterogeneity of the disease has hampered the development of new therapeutic strategies.

Gastric or Esophageal Cancer

An estimated 17650 adult patients in the US will be diagnosed with gastric cancer and approximately 16080 deaths will occur from this disease in 2019 (American Cancer Society, Survival Rates for Esophageal Cancer, 31 Jan. 2019c, https://www.cancer.org/cancer/esophagus-cancer/detection-diagnosis-staging/survival-rates.html, accessed 6 Jun. 2019). An estimated 27510 adults in the US will be diagnosed with esophageal cancer and approximately 11140 deaths will occur from this disease in 2019 (American Cancer Society, Key Statistics About Stomach Cancer, 9 Jan. 2019d, https://www.cancer.org/cancer/stomach-cancer/about/key-statistics.html, accessed 6 Jun. 2019). Rates of esophageal adenocarcinoma and gastric cardia adenocarcinoma have increased, while rates of esophageal squamous cell carcinoma and gastric noncardia adenocarcinoma have decreased, suggesting distinct etiologies (Crew & Neugut, World J Gastroenterol. 2016;354-362).

Chemotherapy can provide a significant decrease in symptoms for patients with unresectable, locally advanced, or metastatic disease. Single agents that produce partial response (PR) rates (cisplatin, doxorubicin, and mitomycin) are considered the most active in gastrointestinal (GI) cancers (Preusser P et al, Oncology 1998;99-102). Combination regimens employing these agents result in higher response rates (30% to 50%) but are associated with a greater degree of toxicity and produce similar OS (ranging from 6 to 10 months), as compared with single-agent therapy (Preusser P et al, Oncology 1998;99-102). The identification of new agents is therefore essential if prolongation of patient survival is to be reached.

There is a significant need for additional therapeutic methods for cancers. These include the use of antibodies and antibody drug conjugates as treatment modalities.

3. Summary

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has hormone receptor positive and human epidermal growth factor receptor 2 negative (HR+/HER2−) breast cancer.

In some embodiments of the methods provided herein, the HR+/HER2− breast cancer is estrogen receptor (ER) positive and/or progesterone receptor (PR) positive, and HER2 negative.

In some embodiments of the methods provided herein, the subject has locally advanced or metastatic cancer.

In some embodiments of the methods provided herein, the subject has previously received at least one line of an endocrine therapy and a cyclin-dependent kinase (CDK) 4/6 inhibitor in metastatic or locally advanced setting.

In some embodiments of the methods provided herein, the subject has previously received a treatment with a taxane or anthracycline.

In some embodiments of the methods provided herein, the subject has a deleterious germline mutation in breast cancer susceptibility gene (BRCA)1 or BRCA2, and wherein the subject has previously been treated with a poly ADP ribose polymerase (PARP) inhibitor.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has ER negative, PR negative, and HER2 negative (ER—/PR—/HER2−) breast cancer.

In some embodiments of the methods provided herein, for example and not by way of limitation, the methods described in the first paragraph before the present paragraph (paragraph [0036]), the subject has locally advanced or metastatic cancer.

In some embodiments of the methods provided herein, the subject has previously received at least two lines of systemic therapies.

In some embodiments of the methods provided herein, the subject has previously received a treatment with a taxane.

In some embodiments of the methods provided herein, for example and not by way of limitation, the methods described in the first to the fourth paragraphs before the present paragraph (paragraphs [0036] to [0039], the subject has a deleterious germline mutation in breast cancer susceptibility gene (BRCA)1 or BRCA2, and wherein the subject has previously been treated with a poly ADP ribose polymerase (PARP) inhibitor.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has squamous non-small cell lung cancer (NSCLC).

In some embodiments of the methods provided herein, for example and not by way of limitation, the methods described in the first paragraph before the present paragraph (paragraph) [0041]), the subject has locally advanced or metastatic cancer.

In some embodiments of the methods provided herein, the subject has progressed or relapsed following a platinum-based therapy.

In some embodiments of the methods provided herein, the subject has progressed or relapsed within 12 months after a platinum-based therapy.

In some embodiments of the methods provided herein, the subject has previously received a therapy with an inhibitor of programmed cell death protein-1 (PD-1) or an inhibitor of programmed cell death-ligand 1 (PD-L1), wherein optionally the inhibitor of PD-1 is nivolumab and wherein optionally the inhibitor of PD-L1 is selected from a group consisting of atezolizumab, avelumab, and durvalumab.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has non-squamous NSCLC.

In some embodiments of the methods provided herein, the subject has wild-type epidermal growth factor receptor (EGFR) and wild-type anaplastic lymphoma kinase (ALK).

In some embodiments of the methods provided herein, for example and not by way of limitation, the methods described in the first to the second paragraphs before the present paragraph (paragraphs [0046] to [0047], the subject has locally advanced or metastatic cancer.

In some embodiments of the methods provided herein, for example and not by way of limitation, the methods described in in the first to the third paragraphs before the present paragraph (paragraphs [0046] to [0048]), the subject has progressed or relapsed following a platinum-based therapy.

In some embodiments of the methods provided herein, for example and not by way of limitation, the methods described in in the first to the fourth paragraphs before the present paragraph (paragraphs [0046] to [0049]), the subject has progressed or relapsed within 12 months after a platinum-based therapy.

In some embodiments of the methods provided herein, for example and not by way of limitation, the methods described in in the first to the fifth paragraphs before the present paragraph (paragraphs [0046] to [0050]), the subject has previously received a therapy with an inhibitor of programmed cell death protein-1 (PD-1) or an inhibitor of programmed cell death-ligand 1 (PD-L1), wherein optionally the inhibitor of PD-1 is nivolumab and wherein optionally the inhibitor of PD-L1 is selected from a group consisting of atezolizumab, avelumab, and durvalumab.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has locally advanced or metastatic head and neck cancer.

In some embodiments of the methods provided herein, for example and not by way of limitation, the methods described in in the first paragraph before the present paragraph (paragraph [0052]), the subject has progressed or relapsed following a platinum-based therapy.

In some embodiments of the methods provided herein, the subject has progressed or relapsed within 6 months after a platinum-based therapy.

In some embodiments of the methods provided herein, for example and not by way of limitation, the methods described in in the first to the third paragraphs before the present paragraph (paragraphs [0052] to [0054]), the subject has previously received a therapy with an inhibitor of programmed cell death protein-1 (PD-1) or an inhibitor of programmed cell death-ligand 1 (PD-L1), wherein optionally the inhibitor of PD-1 is nivolumab and wherein optionally the inhibitor of PD-L1 is selected from a group consisting of atezolizumab, avelumab, and durvalumab.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has gastric or esophageal cancer.

In some embodiments of the methods provided herein, for example and not by way of limitation, the methods described in in the first paragraph before the present paragraph (paragraph [0056]), the subject has locally advanced or metastatic cancer.

In some embodiments of the methods provided herein, the subject has progressed or relapsed following a platinum-based therapy or a chemotherapy that included a fluoropyrimidine.

NAI-1532487283v1

In some embodiments of the methods provided herein, the subject has progressed or relapsed within 6 months after the platinum-based therapy or the chemotherapy that included a fluoropyrimidine.

In some embodiments of the methods provided herein, the gastric or esophageal cancer is HER2 positive cancer, and wherein the subject has previously received a HER2 directed therapy.

In some embodiments of the methods provided herein, the antibody or antigen binding fragment thereof comprises CDR H1 comprising the amino acid sequence of SEQ ID NO:9, CDR H2 comprising the amino acid sequence of SEQ ID NO:10, CDR H3 comprising the amino acid sequence of SEQ ID NO:11; CDR L1 comprising the amino acid sequence of SEQ ID NO:12, CDR L2 comprising the amino acid sequence of SEQ ID NO:13, and CDR L3 comprising the amino acid sequence of SEQ ID NO:14.

In some embodiments of the methods provided herein, the antibody or antigen binding fragment thereof comprises CDR H1 comprising the amino acid sequence of SEQ ID NO:16, CDR H2 comprising the amino acid sequence of SEQ ID NO:17, CDR H3 comprising the amino acid sequence of SEQ ID NO:18; CDR L1 comprising the amino acid sequence of SEQ ID NO:19, CDR L2 comprising the amino acid sequence of SEQ ID NO:20, and CDR L3 comprising the amino acid sequence of SEQ ID NO:21.

In some embodiments of the methods provided herein, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments of the methods provided herein, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments of the methods provided herein, the antigen binding fragment is an Fab, F(ab′)2, Fv or scFv fragment.

In some embodiments of the methods provided herein, the antibody is a fully human antibody.

In some embodiments of the methods provided herein, the antibody or antigen binding fragment thereof is recombinantly produced.

In some embodiments of the methods provided herein, the antibody drug conjugate has the following structure:

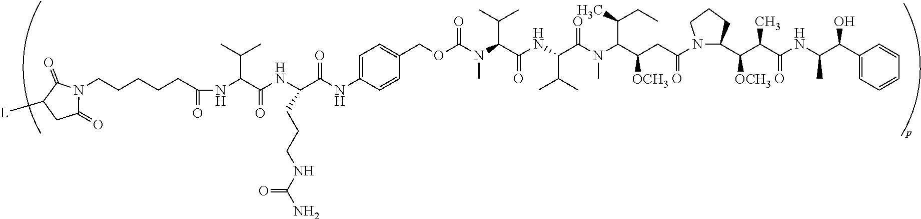

wherein L- represents the antibody or antigen binding fragment thereof and p is from 1 to 10.

In some embodiments of the methods provided herein, p is from 2 to 8.

In some embodiments of the methods provided herein, p is from 3 to 5.

In some embodiments of the methods provided herein, the antibody or antigen binding fragment is linked to each unit of monomethyl auristatin E (M MAE) via a linker.

In some embodiments of the methods provided herein, the linker is an enzyme-cleavable linker, and wherein the linker forms a bond with a sulfur atom of the antibody or antigen binding fragment thereof.

In some embodiments of the methods provided herein, the linker has a formula of:— Aa—Ww—Yy—; wherein —A— is a stretcher unit, a is 0 or 1; -W- is an amino acid unit, w is an integer ranging from 0 to 12; and -Y- is a spacer unit, y is 0, 1, or 2.

In some embodiments of the methods provided herein, the stretcher unit has the structure of Formula (1) below; the amino acid unit is valine citrulline; and the spacer unit is a PAB group comprising the structure of Formula (2) below:

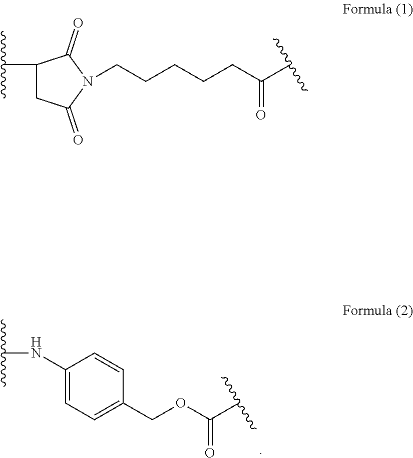 [SMILES: *C(=O)CCCCCN1C(=O)CC(*)C1=O ; *Nc1ccc(COC(*)=O)cc1]

In some embodiments of the methods provided herein, the stretcher unit forms a bond with a sulfur atom of the antibody or antigen binding fragment thereof; and wherein the spacer unit is linked to MMAE via a carbamate group.

In some embodiments of the methods provided herein, the antibody drug conjugate comprises from 1 to 10 units of MMAE per antibody or antigen binding fragment thereof.

In some embodiments of the methods provided herein, the antibody drug conjugate comprises from 2 to 8 units of MMAE per antibody or antigen binding fragment thereof.

In some embodiments of the methods provided herein, the antibody drug conjugate comprises from 3 to 5 units of MMAE per antibody or antigen binding fragment thereof.

In some embodiments of the methods provided herein, the antibody drug conjugate is formulated in a pharmaceutical composition, wherein the pharmaceutical composition comprises a pharmaceutically acceptable excipient comprising L-histidine, polysorbate-20 (TWEEN-20), and at least one of trehalose dihydrate and sucrose.

In some embodiments of the methods provided herein, the pharmaceutical composition comprises the antibody drug conjugate at a concentration of from 1 to 20 mg/mL, from 5 to 15 mg/mL, from 8 to 12 mg/mL.

In some embodiments of the methods provided herein, the antibody drug conjugate is at a concentration of about 10 mg/mL.

In some embodiments of the methods provided herein, the L-histidine is present in the range of 5 to 50 mM, 10 to 40 mM, 15 to 35 mM, 15 to 30 mM, or 15 to 25 mM.

In some embodiments of the methods provided herein, the L-histidine is present at about 20 mM.

In some embodiments of the methods provided herein, the concentration of TWEEN-20 is in the range of from 0.001 to 0.1% (v/v), from 0.0025 to 0.075% (v/v), from 0.005 to 0.05% (v/v), or from 0.01 to 0.03% (v/v).

In some embodiments of the methods provided herein, the concentration of TWEEN-20 is about 0.02% (v/v).

In some embodiments of the methods provided herein, the pharmaceutical composition comprises trehalose dihydrate.

In some embodiments of the methods provided herein, the trehalose dihydrate is present in the range of 1 to 20% (w/v), 2 to 15% (w/v), 3 to 10% (w/v), or 4 to 6% (w/v).

In some embodiments of the methods provided herein, the trehalose dihydrate is present at about 5.5% (w/v).

In some embodiments of the methods provided herein, the trehalose dihydrate is present in the range of 50 mM to 300 mM, 75 mM to 250 mM, 100 mM to 200 mM, or 130 mM to 150 mM.

In some embodiments of the methods provided herein, the trehalose dihydrate is present at about 146 mM.

In some embodiments of the methods provided herein, the pharmaceutical composition comprises sucrose.

In some embodiments of the methods provided herein, the sucrose is present in the range of 1 to 20% (w/v), 2 to 15% (w/v), 3 to 10% (w/v), or 4 to 6% (w/v).

In some embodiments of the methods provided herein, the sucrose is present at about 5.5% (w/v).

In some embodiments of the methods provided herein, the sucrose is present in the range of 50 mM to 300 mM, 75 mM to 250 mM, 100 mM to 200 mM, or 130 mM to 150 mM.

In some embodiments of the methods provided herein, the sucrose is present at about 146 mM.

In some embodiments of the methods provided herein, the pharmaceutical composition has a pH in a range of 5.5 to 6.5 or 5.7 to 6.3.

In some embodiments of the methods provided herein, the pharmaceutical composition has a pH of about 6.0.

In some embodiments of the methods provided herein, the pH is taken at room temperature, at 15° C. to 27° C., at about 4° C., or at about 25° C.

In some embodiments of the methods provided herein, the pharmaceutical composition comprises hydrochloric acid (HCl) or succinic acid.

In some embodiments of the methods provided herein, the pharmaceutical composition comprises about 20 mM L-histidine, about 0.02% (w/v) TWEEN-20, about 5.5% (w/v) trehalose dihydrate, and HCl; and wherein the pH is about 6.0 at 25° C.

In some embodiments of the methods provided herein, the pharmaceutical composition comprises about 20 mM L-histidine, about 0.02% (w/v) TWEEN-20, about 5.5% (w/v) trehalose dihydrate, and succinic acid; and wherein the pH is about 6.0 at 25° C.

In some embodiments of the methods provided herein, the pharmaceutical composition comprises about 20 mM L-histidine, about 0.02% (w/v) TWEEN-20, about 5.5% (w/v) sucrose, and HCl; and wherein the pH is about 6.0 at 25° C.

In some embodiments of the methods provided herein, the pharmaceutical composition comprises about 20 mM L-histidine, about 0.02% (w/v) TWEEN-20, about 5.5% (w/v) sucrose, and succinic acid; and wherein the pH is about 6.0 at 25° C.

In some embodiments of the methods provided herein, wherein the antibody drug conjugate is administered at a dose of 1 to 10 mg/kg of the subject's body weight, 1 to 5 mg/kg of the subject's body weight, 1 to 2.5 mg/kg of the subject's body weight, or 1 to 1.25 mg/kg of the subject's body weight.

In some embodiments of the methods provided herein, the antibody drug conjugate is administered at a dose of about 1 mg/kg of the subject's body weight.

In some embodiments of the methods provided herein, the antibody drug conjugate is administered at a dose of about 1.25 mg/kg of the subject's body weight.

In some embodiments of the methods provided herein, the antibody drug conjugate is administered by an intravenous (IV) injection or infusion.

In some embodiments of the methods provided herein, the antibody drug conjugate is administered by an intravenous (IV) injection or infusion over about 30 minutes twice every three-week cycle.

In some embodiments of the methods provided herein, the antibody drug conjugate is administered by an intravenous (IV) injection or infusion over about 30 minutes on Days 1 and 8 of every three-week cycle.

In some embodiments of the methods provided herein, the antibody drug conjugate is administered by an intravenous (IV) injection or infusion over about 30 minutes three times every four-week cycle.

In some embodiments of the methods provided herein, the antibody drug conjugate formulated in the pharmaceutical composition is administered by an intravenous (IV) injection or infusion over about 30 minutes on Days 1, 8 and 15 of every four-week cycle.

### 4. Brief Description of the Drawings

FIGS. 1A-E depict the nucleotide and amino acid sequences of 191P4D12 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2 depicts the efficacy of Ha22-2(2,4)6.1-vcMMAE in subcutaneous established human lung cancer xenograft AG-L4 in SCID mice. The results show that treatment with Ha22-2(2,4)6.1-vcMMAE significantly inhibited the growth of AG-L4 lung cancer xenografts implanted subcutaneously in nude mice compared to both the treated and untreated control.

FIG. 3 depicts the efficacy of Ha22-2(2,4)6.1-vcMMAE in subcutaneous established human breast cancer xenograft BT-483 in SCID mice. The results show that treatment with Ha22-2(2,4)6.1-vcMMAE significantly inhibited the growth of BT-483 breast tumor xenografts implanted subcutaneously in SCID mice compared to the treated and untreated control ADCs.

FIGS. 4A-H. Detection of 191P4D12 protein in cancer patient specimens by IHC.

FIGS. 4A-B show breast cancer specimens. FIGS. 4C-D show lung cancer specimens. FIGS. 4E-F show esophageal cancer specimens. FIGS. 4G-H show head and neck cancer specimens.

### 5. DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)2 fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)2, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (MA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®QK384, the Biacore®TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (β), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH (β-sheet framework, or one of three hypervariable regions (L1, L2 or L3) within the non-framework region of the antibody VL (β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below Table 1.

TABLE 1

| | Kabat | AbM | Chothia | Contact | IMGT |
|---|---|---|---|---|---|
| CDR L1 | L24--L34 | L24--L34 | L24--L34 | L30--L36 | L27--L38 |
| CDR L2 | L50--L56 | L50--L56 | L50--L56 | L46--L55 | L56--L65 |
| CDR L3 | L89--L97 | L89--L97 | L89--L97 | L89--L96 | L105--L117 |
| CDR H1 | H31--H35B | H26--H35B | H26--H32 . . . 34 | H30--H35B | H27--H38 |
| | (Kabat Numbering) | | | | |
| CDR H1 | H31--H35 | H26--H35 | H26--H32 | H30--H35 | |
| | (Chothia Numbering) | | | | |
| CDR H2 | H50--H65 | H50--H58 | H52--H56 | H47--H58 | H56--H65 |
| CDR H3 | H95--H102 | H95--H102 | H95--H102 | H93--H101 | H105--H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise noted, the term “alkyl” refers to a saturated straight or branched hydrocarbon comprising from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Alkyl groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)2 and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, —SO₃R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains comprising from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2- butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1 butynyl. Alkenyl and alkynyl groups, whether alone or as part of another group, can be optionally substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)2 and —CN, where each R′ is independently selected from —H, —C₁-C₈ alkyl, —C₂-C₈ alkyenl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, —SO₃R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical comprising from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)2 and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, —SO₃R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH═CHCH2-).

Unless otherwise noted, the term “alkynylene” refers to an optionally substituted alkylene group containing at least one carbon-carbon triple bond. Exemplary alkynylene groups include, for example, acetylene (—C═C—), propargyl (—CH2C═C—), and 4-pentynyl (—CH₂CH₂CH₂C═CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO2, —N₃, —NH₂, —NH(R′), —N(R′)2 and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, -503R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or -aryl.

Unless otherwise noted, the term “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group.

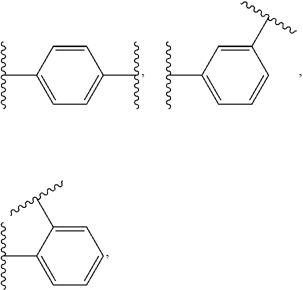

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure. Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960). Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl. Preferred “heterocycle” groups include, but are not limited to, benzofuranyl, benzothiophenyl, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —N₃, —NH₂, —NH(R′), —N(R′)2 and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, —SO₃R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or aryl.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; position 4 of a morpholine; and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo [4,5], [5,5], [5,6] or [6,6] system, or 9 or 10 ring atoms arranged as a bicyclo [5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms. Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)2 and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)2, —NHC(O)R″, —SR″, —SO₃R″, —S(O)₂R″, —S(O)R″, —OH, —N₃, —NH₂, —NH(R″), —N(R″)2 and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and —cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

When a particular group is “substituted”, that group may have one or more substituents, preferably from one to five substituents, more preferably from one to three substituents, most preferably from one to two substituents, independently selected from the list of substituents. The group can, however, generally have any number of substituents selected from halogen. Groups that are substituted are so indicated. It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Protective groups as used herein refer to groups which selectively block, either temporarily or permanently, one reactive site in a multifunctional compound. Suitable hydroxy-protecting groups for use in the present invention are pharmaceutically acceptable and may or may not need to be cleaved from the parent compound after administration to a subject in order for the compound to be active. Cleavage is through normal metabolic processes within the body. Hydroxy protecting groups are well-known in the art, see, Protective Groups in Organic Synthesis by T. W. Greene and P. G. M. Wuts (John Wiley & sons, 3^{rd} Edition) incorporated herein by reference in its entirety and for all purposes and include, for example, ether (e.g., alkyl ethers and silyl ethers including, for example, dialkylsilylether, trialkylsilylether, dialkylalkoxysilylether), ester, carbonate, carbamates, sulfonate, and phosphate protecting groups. Examples of hydroxy protecting groups include, but are not limited to, methyl ether; methoxymethyl ether, methylthiomethyl ether, (phenyldimethylsilyl)methoxymethyl ether, benzyloxymethyl ether, p-methoxybenzyloxymethyl ether, p-nitrobenzyloxymethyl ether, o-nitrobenzyloxymethyl ether, (4-methoxyphenoxy)methyl ether, guaiacolmethyl ether, t-butoxymethyl ether, 4-pentenyloxymethyl ether, siloxymethyl ether, 2-methoxyethoxymethyl ether, 2,2,2-trichloroethoxymethyl ether, bis(2-chloroethoxy)methyl ether, 2-(trimethylsilyl)ethoxymethyl ether, menthoxymethyl ether, tetrahydropyranyl ether, 1-methoxycylcohexyl ether, 4-methoxytetrahydrothiopyranyl ether, 4-methoxytetrahydrothiopyranyl ether S,S-Dioxide, 1-[(2-choro-4-methyl)phenyl]-4-methoxypiperidin-4-yl ether, 1-(2-fluorophneyl)-4-methoxypiperidin-4-yl ether, 1,4-dioxan-2-yl ether, tetrahydrofuranyl ether, tetrahydrothiofuranyl ether; substituted ethyl ethers such as 1-ethoxyethyl ether, 1-(2-chloroethoxy)ethyl ether, 1[2-(trimethylsilyl)ethoxy]ethyl ether, 1-methyl- 1-methoxyethyl ether, 1-methyl- 1-benzyloxyethyl ether, 1-methyl- 1-benzyloxy-2-fluoroethyl ether, 1-methyl-1phenoxyethyl ether, 2-trimethylsilyl ether, t-butyl ether, allyl ether, propargyl ethers, p-chlorophenyl ether, p-methoxyphenyl ether, benzyl ether, p-methoxybenzyl ether 3,4-dimethoxybenzyl ether, trimethylsilyl ether, triethylsilyl ether, tripropylsilylether, dimethylisopropylsilyl ether, diethylisopropylsilyl ether, dimethylhexylsilyl ether, t-butyldimethylsilyl ether, diphenylmethylsilyl ether, benzoylformate ester, acetate ester, chloroacetate ester, dichloroacetate ester, trichloroacetate ester, trifluoroacetate ester, methoxyacetate ester, triphneylmethoxyacetate ester, phenylacetate ester, benzoate ester, alkyl methyl carbonate, alkyl 9-fluorenylmethyl carbonate, alkyl ethyl carbonate, alkyl 2,2,2,-trichloroethyl carbonate, 1,1,-dimethyl-2,2,2-trichloroethyl carbonate, alkylsulfonate, methanesulfonate, benzylsulfonate, tosylate, methylene acetal, ethylidene acetal, and t-butylmethylidene ketal. Preferred protecting groups are represented by the formulas —R^{a}, —Si(R^{a})(R^{a})(R^{a})—C(O)R^{a}, —C(O)OR^{a}, —C(O)NH(R^{a}), —S(O)₂R^{a}, —S(O)₂0 H, P(O)(OH)2, and —P(O)(OH)OR^{a}, wherein R^{a} is C₁-C₂₀ alkyl, C₂-C₂o alkenyl, C₂-C₂o alkynyl, —C₁-C₂o alkylene(carbocycle), —C₂-C₂₀ alkenylene(carbocycle), —C₂-C₂₀ alkynylene(carbocycle), —C₆-C₁₀ aryl, —C₁-C₂o alkylene(aryl), —C₂-C₂o alkenylene(aryl), —C₂-C₂o alkynylene(aryl), —C₁-C₂o alkylene(heterocycle), —C₂-C₂₀ alkenylene(heterocycle), or —C₂-C₂₀ alkynylene(heterocycle) wherein said alkyl, alkenyl, alkynyl, alkylene, alkenylene, alkynylene, aryl, carbocycle, and heterocycle radicals whether alone or as part of another group are optionally substituted.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

The term “compound” refers to and encompasses the chemical compound itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid substitution matrix (block substitution
matrix). The higher the value, the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, Md. (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and)(BLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the)(BLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of)(BLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, sm¹⁵³, Bi²¹²or ²¹³,P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

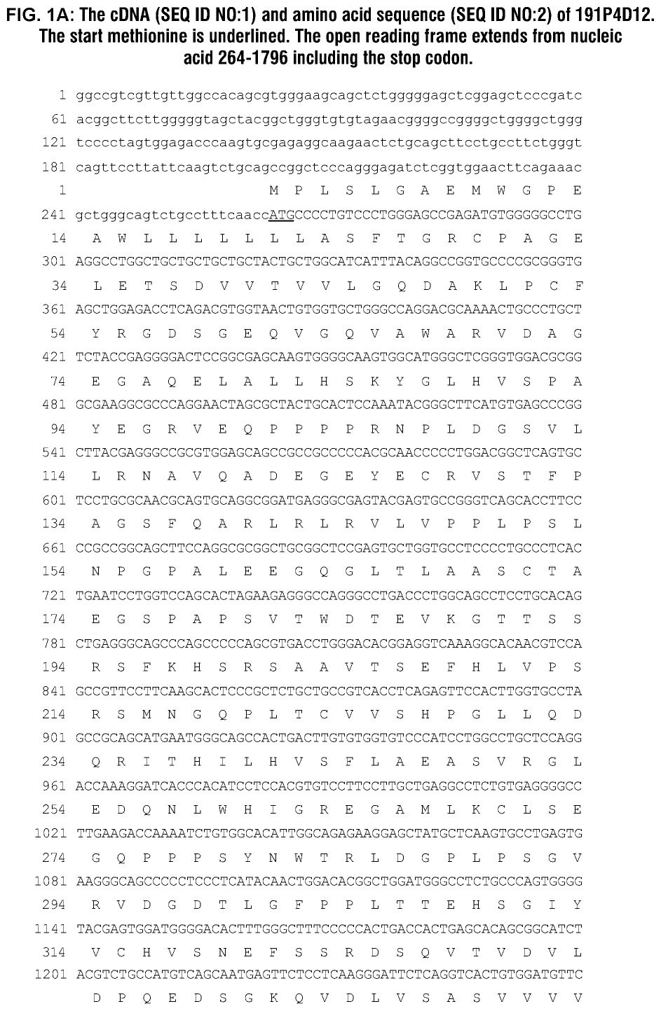

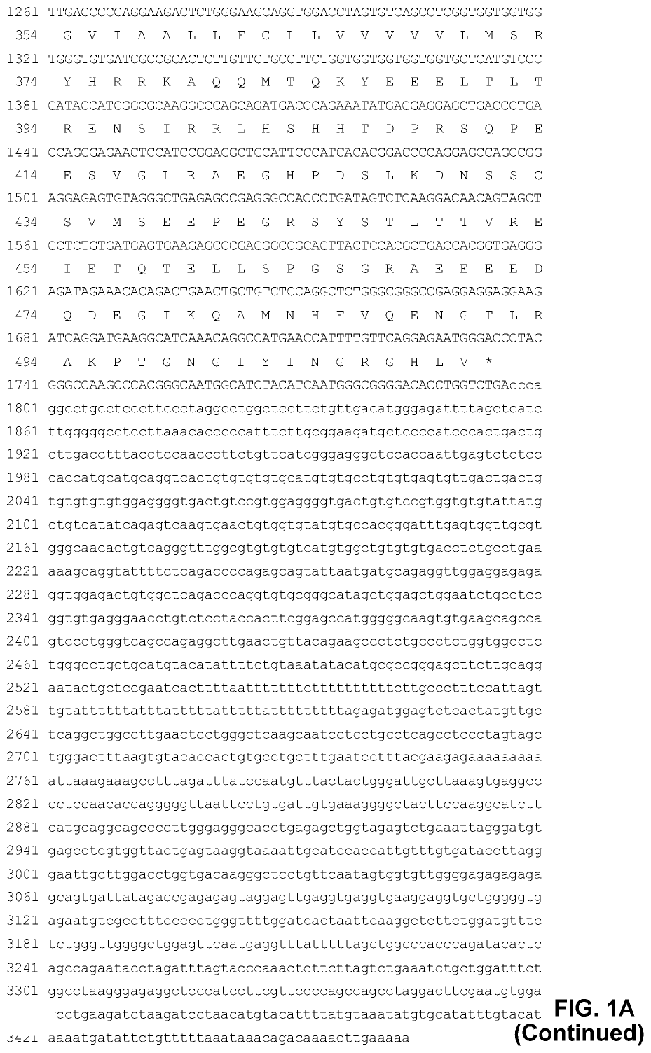

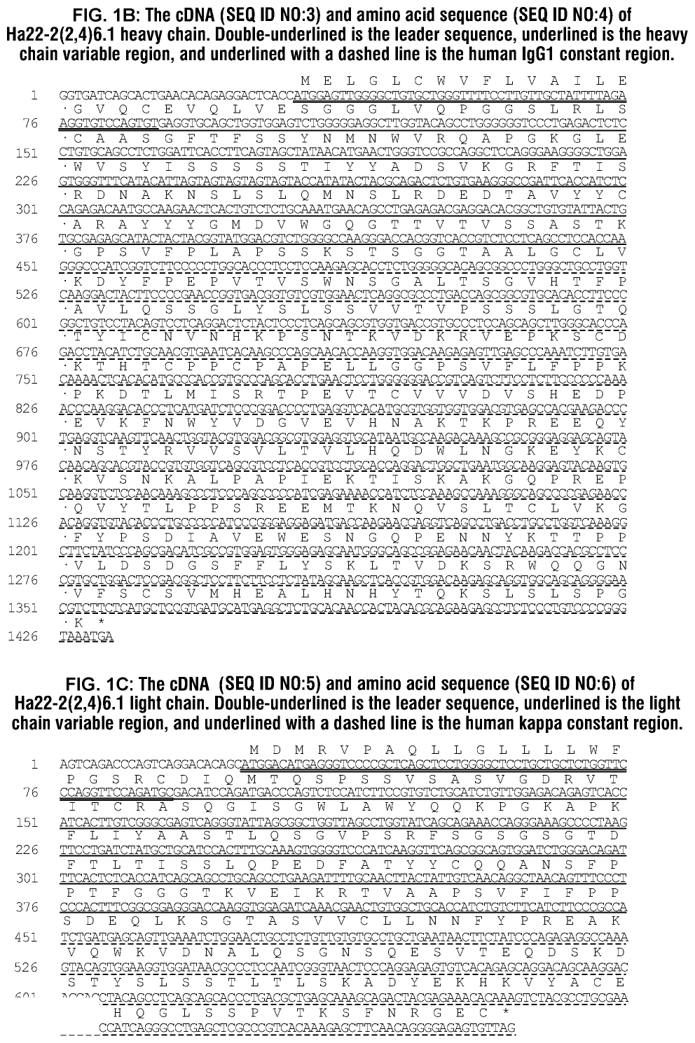

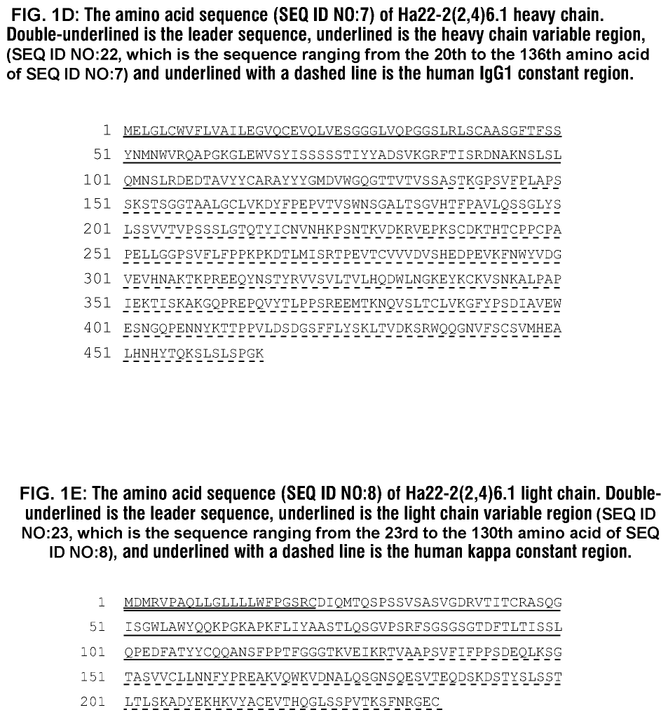

The term “variant” refers to a molecule that exhibits a variation from a described type or norm, such as a protein that has one or more different amino acid residues in the corresponding position(s) of a specifically described protein (e.g. the 191P4D12 protein shown in FIG. 1.) An analog is an example of a variant protein. Splice isoforms and single nucleotides polymorphisms (SNPs) are further examples of variants.

The “191P4D12 proteins” and/or “191P4D12 related proteins” of the invention include those specifically identified herein (see, FIG. 1), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the invention, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids.

5.2 Methods of Treating Cancer

Provided herein are methods for the treatment of various cancers, including breast cancer (e.g., HER+/HER2− breast cancer and triple negative breast cancer that is ER negative, PR negative, and HER2 negative (ER−/PR−/HER2−)), lung cancer (e.g., squamous lung cancer, non-squamous lung cancer, squamous non-small cell lung cancer, and non-squamous non-small cell lung cancer), head and neck cancer, and gastric or esophageal cancer using an antibody drug conjugate (ADC) that binds 191P4D12. In some embodiments the ADC is enfortumab vedotin (also known as anti-191P4D12-ADC, Ha22-2(2,4)6.1vcMMAE, ASG-22CE, or AGS-22M6E).

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has hormone receptor positive and human epidermal growth factor receptor 2 negative (HR+/HER2−) breast cancer.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has ER negative, PR negative, and HER2 negative (ER—/PR—/HER2−) breast cancer.

In another aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has squamous non-small cell lung cancer (NSCLC).

In a further aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has non-squamous NSCLC.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has locally advanced or metastatic head and neck cancer.

In certain aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has gastric or esophageal cancer.

In all the methods provided herein and specifically those described in the previous seven paragraphs (paragraphs [00188] to [00194]): the therapeutic agents that can be used are described in Section 5.3, selection of patients for treatment is described herein and exemplified in Section 5.2 and Section 6, dosing regimens and pharmaceutical composition for administering the therapeutic agent are described in Section 5.4 and Section 6 below, the biomarkers that can be used for identifying the therapeutic agents, selecting the patients, determining the outcome of these methods, and/or serving as criteria in any way for these methods are described herein and exemplified in Section 5.2 and Section 6, therapeutic outcomes for the methods provided herein can be improvement of the biomarkers described herein, for example, those described and exemplified in in Section 5.2 and Section 6. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for the treatment of breast cancer in a subject. In some embodiments, the breast cancer is hormone receptor positive and human epidermal growth factor receptor 2 negative (HR+/HER2−) breast cancer. In some embodiments, the breast cancer is estrogen receptor (ER) positive and/or progesterone receptor (PR) positive, and HER2 negative. In some embodiments, the breast cancer is ER positive, PR positive, and HER2 negative. In some embodiments, the breast cancer is ER positive and HER2 negative. In some embodiments, the breast cancer is PR positive and HER2 negative. In some embodiments, the breast cancers, including for example, the HR+/HER2− breast cancer, the ER positive, PR positive, and HER2 negative breast cancer, the ER positive and HER2 negative breast cancer, the PR positive and HER2 negative breast cancer are confirmed histologically, cytologically, or both histologically and cytologically. In some embodiments, the histological, cytological, or both histological and cytological confirmation are conducted per American Society of Clinical Oncology/College of American Pathologists (ASCO/CAP) guidelines based on the most recently analyzed tissue.

In some embodiments, the hormone receptor positive and human epidermal growth factor receptor 2 negative (HR+/HER2−) breast cancer is locally advanced or metastatic breast cancer. In some embodiments, the ER positive and/or progesterone receptor (PR) positive, and HER2 negative breast cancer is locally advanced or metastatic breast cancer. In some embodiments, the ER positive, PR positive, and HER2 negative breast cancer is locally advanced or metastatic breast cancer. In some embodiments, the ER positive and HER2 negative breast cancer is locally advanced or metastatic breast cancer. In some embodiments, the PR positive and HER2 negative breast cancer is locally advanced or metastatic breast cancer. In some embodiments, the locally advanced or metastatic breast cancers, including for example, the HR+/HER2− breast cancer, the ER positive, PR positive, and HER2 negative breast cancer, the ER positive and HER2 negative breast cancer, the PR positive and HER2 negative breast cancer are confirmed histologically, cytologically, or both histologically and cytologically. In some embodiments, such histological, cytological, or both histological and cytological confirmation are conducted per American Society of Clinical Oncology/College of American Pathologists (ASCO/CAP) guidelines based on the most recently analyzed tissue.

In some embodiments, subjects having breast cancer and treated with the methods provided herein have received ≥1 line of endocrine therapy and a cyclin-dependent kinase (CDK) 4/6 inhibitor in the metastatic or locally advanced setting. In some embodiments, subjects having breast cancer and treated with the methods provided herein have received prior treatment with a taxane or anthracycline in any setting. In some embodiments, subjects having breast cancer and treated with the methods provided herein have a deleterious germline mutation in breast cancer susceptibility gene (BRCA)1 or 2 must have been treated with a poly ADP ribose polymerase (PARP) inhibitor.

In some specific embodiments, subjects treated with the methods provided herein have histologically or cytologically-confirmed HR+/HER2− breast cancers that are defined as ER positive and/or progesterone receptor (PR) positive, and HER2 negative as per American Society of Clinical Oncology/College of American Pathologists (ASCO/CAP) guidelines based on the most recently analyzed tissue; have a locally advanced or metastatic disease; have received ≥1 line of endocrine therapy and a cyclin-dependent kinase (CDK) 4/6 inhibitor in the metastatic or locally advanced setting; have received prior treatment with a taxane or anthracycline in any setting; and/or have a deleterious germline mutation in breast cancer susceptibility gene (BRCA)1 or 2 must have been treated with a poly ADP ribose polymerase (PARP) inhibitor.

In certain embodiments, the methods provided herein are used for the treatment of triple negative breast cancer (TNBC) in a subject. In some embodiments, the TNBC is histologically and/or cytologically-confirmed TNBC. In some embodiments, the TNBC is determined according to TNBC histology (ER-negative/PR-negative/HER2−negative) as per ASCO/CAP guidelines based on the most recently analyzed tissue. In some embodiments, the TNBC is locally advanced or metastatic. In some embodiments, subjects having TNBC and treated with the methods provided herein have had ≥2 lines of systemic therapy. In some embodiments, subjects having TNBC and treated with the methods provided herein have had ≥2 lines of systemic therapy, including a taxane in any setting. In some embodiments, subjects having TNBC and treated with the methods provided herein have a deleterious germline mutation in BRCA1, BRCA2, or both BRCA1 and BRCA2. In some embodiments, subjects having TNBC and treated with the methods provided herein have been treated with a PARP inhibitor. In some embodiments, the subjects treated by the methods provided herein for TNBC have any permutation or combination of the characteristics described in this paragraph.

In some specific embodiments, subjects treated with the methods provided herein have histologically or cytologically-confirmed TNBC that is defined as unequivocal TNBC histology (ER-negative/PR-negative/HER2−negative) as per ASCO/CAP guidelines based on the most recently analyzed tissue; have locally advanced or metastatic disease; have had ≥2 lines of systemic therapy, including a taxane in any setting; have had a deleterious germline mutation in BRCA/or BRCA2 or both; and/or have been treated with a PARP inhibitor.

In certain embodiments, the methods provided herein are used for the treatment of squamous non-small cell lung cancer (NSCLC) in a subject. In some embodiments, the squamous NSCLC is histologically- and/or cytologically-confirmed squamous NSCLC. In some embodiments, the squamous NSCLC is locally advanced or metastatic. In some embodiments, subjects having squamous NSCLC and treated with the methods provided herein have progressed or relapsed following platinum-based therapy, including e.g. platinum therapy administered in the adjuvant setting if relapse occurred within 12 months after completion. In some embodiments, subjects having squamous NSCLC and treated with the methods provided herein have received prior therapy with an anti-programmed cell death protein-1 (PD-1) or anti-programmed cell death-ligand 1 (PD-L1) if eligible based on subject's tumor PD-1 or PD-L1 expression and local treatment guidelines.

In some specific embodiments, subjects treated with the methods provided herein have histologically- or cytologically-confirmed squamous NSCLC; have a locally advanced or metastatic disease; have progressed or relapsed following platinum-based therapy including e.g. platinum therapy administered in the adjuvant setting counts as a regimen if relapse occurred within 12 months after completion; and/or have received prior therapy with an anti-programmed cell death protein-1 (PD-1) or anti-programmed cell death-ligand 1 (PD-L1) if eligible based on subject's tumor PD-1 or PD-L1 expression and local treatment guidelines.

In certain embodiments, the methods provided herein are used for the treatment of non-squamous NSCLC in a subject. In some embodiments, the squamous NSCLC is histologically-and/or cytologically-confirmed squamous NSCLC. In some embodiments, the squamous NSCLC is epidermal growth factor receptor (EGFR) wild type and anaplastic lymphoma kinase (ALK) wild type. In some embodiments, the squamous NSCLC is EGFR wild type and ALK wild type by local laboratory standards. In some embodiments, the non-squamous NSCLC is locally advanced or metastatic. In some embodiments, subjects having squamous NSCLC and treated with the methods provided herein have progressed or relapsed following platinum-based therapy in the metastatic or locally advanced setting including e.g. platinum therapy administered in the adjuvant setting if relapse occurred within 12 months after completion. In some embodiments, subjects having squamous NSCLC and treated with the methods provided herein have received an anti-PD-1 or anti-PD-L1 therapy if eligible based on subject's tumor PD-1 or PD-L1 expression and local treatment guidelines.

In some specific embodiments, subjects treated with the methods provided herein have histologically or cytologically-confirmed non-squamous NSCLC that is EGFR wild type and ALK wild type by local laboratory standards; have locally advanced or metastatic disease; have progressed or relapsed following platinum-based therapy in the metastatic or locally advanced setting including e.g. platinum therapy administered in the adjuvant setting if relapse occurred within 12 months after completion; have received an anti-PD-1 or anti-PD-L1 therapy if eligible based on subject's tumor PD-1 or PD-L1 expression and local treatment guidelines.

In certain embodiments, the methods provided herein are used for the treatment of head and neck cancer in a subject. In some embodiments, the head and neck cancer is histologically-and/or cytologically-confirmed head and neck cancer. In some embodiments, the head and neck cancer is locally advanced or metastatic. In some embodiments, subjects having head and neck cancer and treated with the methods provided herein have progressed or relapsed following platinum containing regimen in the metastatic or locally advanced setting, which platinum containing regimen does not include platinum regimens administered as part of multimodal therapy in the curative setting unless the subject relapsed or progressed within 6 months after completion. In some embodiments, subjects having head and neck cancer and treated with the methods provided herein have received an anti-PD-1 or anti-PD-L1 therapy if eligible based on subject's tumor PD-1 or PD-L1 expression and local treatment guidelines.

In some specific embodiments, subjects treated with the methods provided herein have histologically- or cytologically-confirmed head and neck cancer; have a locally advanced or metastatic disease; have progressed or relapsed following platinum containing regimen in the metastatic or locally advanced setting, which does not include platinum regimens administered as part of multimodal therapy in the curative setting unless the subject relapsed or progressed within 6 months after completion; have received an anti-PD-1 or anti-PD-L1 therapy if eligible based on subject's tumor PD-1 or PD-L1 expression and local treatment guidelines.

In certain embodiments, the methods provided herein are used for the treatment of gastric or esophageal cancer in a subject. In some embodiments, the gastric or esophageal cancer is histologically- and/or cytologically-confirmed gastric or esophageal cancer. In some embodiments, the gastric or esophageal cancer is locally advanced or metastatic. In some embodiments, subjects having head and neck cancer and treated with the methods provided herein have progressed or relapsed following chemotherapy regimens that included a fluoropyrimidine and a platinum for metastatic disease or locally advanced disease, which chemotherapy regimens do not include neoadjuvant or adjuvant regimens unless the subject relapsed or progressed within 6 months after completion. In some embodiments, subjects having head and neck cancer and treated with the methods provided herein have received HER2 directed therapy if the subjects have HER2 positive cancer. In some embodiments, subjects having head and neck cancer and treated with the methods provided herein have HER2 positive cancer and have received HER2 directed therapy.

In some specific embodiments, subjects treated with the methods provided herein have histologically- or cytologically-confirmed gastric or esophageal cancer; have locally advanced or metastatic disease; have progressed or relapsed following chemotherapy regimens that included a fluoropyrimidine and a platinum for metastatic disease or locally advanced disease, which chemotherapy regimens do not include neoadjuvant or adjuvant regimens unless the subject relapsed or progressed within 6 months after completion; have HER2 positive cancer and have received HER2 directed therapy. In another specific embodiments, subjects treated with the methods provided herein have histologically- or cytologically-confirmed gastric or esophageal cancer; have locally advanced or metastatic disease; have progressed or relapsed following chemotherapy regimens that included a fluoropyrimidine and a platinum for metastatic disease or locally advanced disease, which chemotherapy regimens do not include neoadjuvant or adjuvant regimens unless the subject relapsed or progressed within 6 months after completion.

In certain embodiments, the methods provided herein are used for treating subjects having cancers that express 191P4D12 RNA, express 191P4D12 protein, or express both 191P4D12 RNA and 191P4D12 protein. In certain embodiments, the methods provided herein are used for treating subjects having cancers that express both 191P4D12 RNA and 191P4D12 protein, including for example, squamous NSCLC, non-squamous NSCLC, Gastric (GEJ) cancer, esophageal cancer, HNSCC, NSCLC-adenocarcinoma, head and neck cancer (e.g. head & neck cancer-squamous), and breast cancer (including HR+/HER2− breast cancer and TNBC). In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by 2 methods of IHC.

In certain embodiments, the methods provided herein are used for treating subjects having cancers, wherein the cancers express 191P4D12 RNA, express 191P4D12 protein, or express both 191P4D12 RNA and 191P4D12 protein, and wherein the cancers are sensitive to cytotoxic agents (such as Vinca and MMAE) blocking microtubule polymerization. In certain embodiments, the methods provided herein are used for treating subjects having cancers that express both 191P4D12 RNA and 191P4D12 protein and that are sensitive to cytotoxic agents (such as Vinca and MMAE) blocking microtubule polymerization, which cancers include for example, squamous NSCLC, non-squamous NSCLC, Gastric (GEJ) cancer, esophageal cancer, HNSCC, NSCLC-adenocarcinoma, head and neck cancer (e.g. head & neck cancer-squamous), and breast cancer (including HR+/HER2− breast cancer and TNBC).

In some embodiments, the subjects that can be treated in the methods provided herein are subjects having solid tumors, including, for example, subjects having hormone receptor positive and human epidermal growth factor receptor 2 negative (HR+/HER2−) breast cancer, subjects having ER negative, PR negative, and HER2 negative (ER—/PR—/HER2−) breast cancer, subjects having NSCLC, subjects having non-squamous NSCLC, subjects having head cancer, subjects having neck cancer, subjects having head and neck cancer, subjects having gastric cancer, subjects having esophageal cancer, and/or subjects having gastric or esophageal cancer.

In certain embodiments, the subjects that can be treated in the methods provided herein further include subjects having solid tumors that are locally advanced, metastatic (including metastatic malignant), and both locally adcanced and metastatic solid tumors. In some embodiments, the solid tumors that can be treated in the methods provided herein are locally advanced HR+/HER2− breast cancer, locally advanced ER—/PR—/HER2− breast cancer, locally advanced NSCLC, locally advanced non-squamous NSCLC, locally advanced head cancer, locally advanced neck cancer, locally advanced head and neck cancer, locally advanced gastric cancer, locally advanced esophageal cancer, and/or locally advanced gastric and esophageal cancer. In other embodiments, the solid tumors that can be treated in the methods provided herein are metastatic (including malignant or metastatic malignant) HR+/HER2− breast cancer, metastatic (including malignant or metastatic malignant) ER—/PR—/HER2− breast cancer, metastatic (including malignant or metastatic malignant) NSCLC, metastatic (including malignant or metastatic malignant) non-squamous NSCLC, metastatic (including malignant or metastatic malignant) head cancer, metastatic (including malignant or metastatic malignant) neck cancer, metastatic (including malignant or metastatic malignant) head and neck cancer, metastatic (including malignant or metastatic malignant) gastric cancer, metastatic (including malignant or metastatic malignant) esophageal cancer, and/or metastatic (including malignant or metastatic malignant) gastric and esophageal cancer.

In some embodiments, the locally advanced, metastatic (including metastatic malignant), and both locally adcanced and metastatic solid tumors are confirmed histologically, cytologically, or both histologically and cytologically.

In some embodiments, the subjects that can be treated in the methods provided herein progressed or relapsed following one or more other treatments for cancer. The one or more treatments, following which the subjects have progressed or relapsed, include, for example, one or more lines of an endocrine therapy, a cyclin-dependent kinase (CDK) 4/6 inhibitor (including in metastatic or locally advanced setting), a treatment with taxane, a treatment with anthracycline, a poly ADP ribose polymerase (PARP) inhibitor, a platinum-based therapy, a therapy with an inhibitor of programmed cell death protein-1 (PD-1), an inhibitor of programmed cell death-ligand 1 (PD-L1), a chemotherapy that included a fluoropyrimidine, a HER2 directed therapy, and/or any permutation or combination of two or more of the therapies provided in this paragraph and those described herein.

In certain embodiments, the subjects that can be treated in the methods provided herein have previously received at least two, three, four, five, or six lines of systemic therapies. Such systemic therapies can be any treatment using substances that travel through the bloodstream, reaches and affects cells all over the body. Such systemic therapies can be those described in the previous paragraph (paragraph [00215]). In one embodiment, such systemic therapy is a taxane.

In certain embodiments, the subjects that can be treated in the methods provided herein have progressed or relapsed other treatments for cancer within 1, 2, 3, 4, 5, 6, 7, 8, 9, 10, 11, 12, 13, 14, 15, 16, 17, 18, 19, 20, 21, 22, 23, or 24 month after the other treatments, including for example and not by way of limitation, any or any combination of the treatments described in the second paragraph before this present paragraph (paragraph [00215]). In some particular embodiment, the subjects have progressed or relapsed within 6 months after the platinum-based therapy or the chemotherapy that included a fluoropyrimidine. In other particular embodiments, the subjects have progressed or relapsed within 6 months after a platinum-based therapy. In further embodiments, the subjects have progressed or relapsed within 12 months after a platinum-based therapy.

In some embodiments, the subjects that can be treated in the methods provided herein have already received one or more other treatments for cancer. The one or more treatments that the subjects have received, include, for example, one or more lines of an endocrine therapy, a cyclin-dependent kinase (CDK) 4/6 inhibitor (including in metastatic or locally advanced setting), a treatment with a taxane, a treatment with anthracycline, a poly ADP ribose polymerase (PARP) inhibitor, a platinum-based therapy, a therapy with an inhibitor of programmed cell death protein-1 (PD-1), an inhibitor of programmed cell death-ligand 1 (PD-L1), a chemotherapy that included a fluoropyrimidine, a HER2 directed therapy, and/or any permutation or combination of two or more of the therapies provided in this paragraph and those described herein.

In some embodiments, the subjects that can be treated in the methods provided herein have any combination or permutation of having received one or more other treatments for cancer as described in the previous paragraph (paragraph [00218]) and having progressed or relapsed following one or more other treatments for cancer as described in the fourth paragraph before this present paragraph (paragraph [00215]).

In some embodiments, the subjects that can be treated in the methods provided herein have certain phenotypic or genotypic characteristics. In one embodiment, the subjects have HR+/HER2− breast cancer that is also estrogen receptor (ER) positive and HER2 negative. In one embodiment, the subjects have HR+/HER2− breast cancer that is also progesterone receptor (PR) positive and HER2 negative. In one embodiment, the subjects have HR+/HER2− breast cancer that is also estrogen receptor (ER) positive, progesterone receptor (PR) positive and HER2 negative. In one embodiment, the subjects have a deleterious germline mutation in breast cancer susceptibility gene (BRCA)1, BRCA2, or both BRCA1 and BRCA2. In one embodiment, the subjects have ER negative, PR negative, and HER2 negative (ER—/PR—/HER2−) breast cancer. In one embodiment, the subjects have wild-type epidermal growth factor receptor (EGFR). In one embodiment, the subjects have wild-type anaplastic lymphoma kinase (ALK). In one embodiment, the subjects have both wild-type epidermal growth factor receptor (EGFR) and wild-type anaplastic lymphoma kinase (ALK). In some embodiments, the subjects have any permutation and combination of the phenotypic or genotypic characteristics described herein.

In some embodiments, the phenotypic or genotypic characteristics are determined histologically, cytologically, or both histologically and cytologically. In one embodiment, the HR+/HER2− breast cancer that is also estrogen receptor (ER) positive and HER2 negative are determined histologically, cytologically, or both histologically and cytologically. In one embodiment, the HR+/HER2− breast cancer that is also progesterone receptor (PR) positive and HER2 negative are determined histologically, cytologically, or both histologically and cytologically. In one embodiment, the HR+/HER2− breast cancer that is also estrogen receptor (ER) positive, progesterone receptor (PR) positive and HER2 negative are determined histologically, cytologically, or both histologically and cytologically. In one embodiment, the deleterious germline mutation in breast cancer susceptibility gene (BRCA)1, BRCA2, or both BRCA1 and BRCA2 are determined histologically, cytologically, or both histologically and cytologically. In one embodiment, the ER negative, PR negative, and HER2 negative (ER—/PR—/HER2−) breast cancer are determined histologically, cytologically, or both histologically and cytologically. In one embodiment, the wild-type epidermal growth factor receptor (EGFR) are determined histologically, cytologically, or both histologically and cytologically. In one embodiment, the wild-type anaplastic lymphoma kinase (ALK) are determined histologically, cytologically, or both histologically and cytologically. In one embodiment, both wild-type epidermal growth factor receptor (EGFR) and wild-type anaplastic lymphoma kinase (ALK) are determined histologically, cytologically, or both histologically and cytologically.

In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference.

In some embodiments, the phenotypic or genotypic characteristics are determined by sequencing including the next generation sequencing (e.g. NGS from Illumina, Inc), DNA hybridization, and/or RNA hybridization. In one embodiment, the HR+/HER2− breast cancer that is also estrogen receptor (ER) positive and HER2 negative are determined by sequencing including the next generation sequencing (e.g. NGS from Illumina, Inc), DNA hybridization, and/or RNA hybridization. In one embodiment, the HR+/HER2− breast cancer that is also progesterone receptor (PR) positive and HER2 negative are determined by sequencing including the next generation sequencing (e.g. NGS from Illumina, Inc), DNA hybridization, and/or RNA hybridization. In one embodiment, the HR+/HER2− breast cancer that is also estrogen receptor (ER) positive, progesterone receptor (PR) positive and HER2 negative are determined by sequencing including the next generation sequencing (e.g. NGS from Illumina, Inc), DNA hybridization, and/or RNA hybridization. In one embodiment, the deleterious germline mutation in breast cancer susceptibility gene (BRCA)1, BRCA2, or both BRCA1 and BRCA2 are determined by sequencing including the next generation sequencing (e.g. NGS from Illumina, Inc), DNA hybridization, and/or RNA hybridization. In one embodiment, the ER negative, PR negative, and HER2 negative (ER—/PR—/HER2−) breast cancer are determined by sequencing including the next generation sequencing (e.g. NGS from Illumina, Inc), DNA hybridization, and/or RNA hybridization. In one embodiment, the wild-type epidermal growth factor receptor (EGFR) are determined by sequencing including the next generation sequencing (e.g. NGS from Illumina, Inc), DNA hybridization, and/or RNA hybridization. In one embodiment, the wild-type anaplastic lymphoma kinase (ALK) are determined by sequencing including the next generation sequencing (e.g. NGS from Illumina, Inc), DNA hybridization, and/or RNA hybridization. In one embodiment, both wild-type epidermal growth factor receptor (EGFR) and wild-type anaplastic lymphoma kinase (ALK) are determined by sequencing including the next generation sequencing (e.g. NGS from Illumina, Inc), DNA hybridization, and/or RNA hybridization.

In some embodiments, the one or more other treatments for cancer, which the subjects have received or from which the cancers of the subjects have progressed or relapsed, are a PD-1 inhibitor or a PD-L1 inhibitor. In certain embodiments, the PD-1 inhibitor is pembrolizumab or nivolumab. In other embodiments, the PD-L1 inhibitor is selected from a group consisting of atezolizumab, avelumab, and durvalumab. Other examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In certain embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™ In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In further embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In some embodiments, the subjects that can be treated in the methods provided herein is a mammal. In some embodiments, the subjects that can be treated in the methods provided herein is a human.

5.3 Anti-191P4D12 Antibody Drug Conjugate

In general the methods provided herein utilize an anti-191P4D12 ADC described herein and/or in U.S. Pat. No. 8,637,642, which is herein incorporated in its entirety by reference. The anti-191P4D12 antibody drug conjugate provided herein comprises an antibody or antigen binding fragment thereof that binds to 191P4D12 conjugated to one or more units of cytotoxic agents (or drug units). The cytotoxic agents (or drug units) can be covalently linked directly or via a linker unit (LU).

In some embodiments, the antibody drug conjugate compound has the following formula:

L−(LU-D)ₚ (I)

or a pharmaceutically acceptable salt or solvate thereof; wherein:

L is the antibody unit, e.g., the anti-191P4D12 antibody or an antigen binding fragment thereof as provided in Section 5.3.1 below, and

(LU-D) is a linker unit-drug unit moiety, wherein:

LU- is a linker unit, and

D is a drug unit having cytostatic or cytotoxic activity against a target cell; and

p is an integer from 1 to 20.

In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10.

In some embodiments, the antibody drug conjugate compound has the following formula:

L−(Aₐ-W_{w}-Y_{y}-D)ₚ (II)

or a pharmaceutically acceptable salt or solvate thereof, wherein:

L is the Antibody unit, e.g., the anti-191P4D12 antibody or an antigen binding fragment thereof as provided in Section 5.3.1 below; and

-Aₐ-W_{w}-Y_{y}- is a linker unit (LU), wherein:

-A- is a stretcher unit,

a is 0 or 1,

each -W- is independently an amino acid unit,

w is an integer ranging from 0 to 12,

-Y- is a self-immolative spacer unit,

y is 0, 1 or 2;

D is a drug units having cytostatic or cytotoxic activity against the target cell; and

p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 8, 2 to 7, 2 to 6, 2 to 5, 2 to 4 or 2 to 3. In other embodiments, p is 1, 2, 3, 4, 5 or 6. In some embodiments, p is 2 or 4. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

For compositions comprising a plurality antibodies or antigen binding fragments thereof, the drug loading is represented by p, the average number of drug molecules per antibody unit. Drug loading may range from 1 to 20 drugs (D) per antibody. The average number of drugs per antibody in preparation of conjugation reactions may be characterized by conventional means such as mass spectroscopy, ELISA assay, and HPLC. The quantitative distribution of antibody drug conjugates in terms of p may also be determined. In some instances, separation, purification, and characterization of homogeneous antibody drug conjugates where p is a certain value from antibody drug conjugates with other drug loadings may be achieved by means such as reverse phase HPLC or electrophoresis. In exemplary embodiments, p is from 2 to 8.

5.3.1 Anti-191P4D12 Antibodies or Antigen Binding Fragments

In one embodiment, the antibody or antigen binding fragment thereof that binds to 191P4D12-related proteins is an antibody or antigen binding fragment that specifically binds to 191P4D12 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to 191P4D12 protein comprising amino acid sequence of SEQ ID NO:2 includes antibodies that can bind to other 191P4D12-related proteins. For example, antibodies that bind 191P4D12 protein comprising amino acid sequence of SEQ ID NO:2 can bind 191P4D12-related proteins such as 191P4D12 variants and the homologs or analogs thereof.

In some embodiments, the anti-191P4D12 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIGS. 1B and 1C.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). SEQ ID NO: 22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMN
WVRQAPGKGLEWVSYISSSSSTIYYADSVKGRFTI
SRDNAKNSLSLQMNSLRDEDTAVYYCARAYYYGMD
VWGQGTTVTVSS
SEQ ID NO: 23
DIQMTQSPSSVSASVGDRVTITCRASQGISGWLAW
YQQKPGKAPKFLIYAASTLQSGVPSRFSGSGSGTD
FTLTISSLQPEDFATYYCQQANSFPPTFGGGTKVE
IKR
SEQ ID NO: 7
MELGLCWVFLVAILEGVQCEVQLVESGGGLVQPGG
SLRLSCAASGFTFSSYNMNWVRQAPGKGLEWVSYI
SSSSSTIYYADSVKGRFTISRDNAKNSLSLQMNSL
RDEDTAVYYCARAYYYGMDVWGQGTTVTVSSASTK
GPSVFPLAPSSKSTSGGTAALGCLVKDYFPEPVTV
SWNSGALTSGVHTFPAVLQSSGLYSLSSVVTVPSS
SLGTQTYICNVNHKPSNTKVDKRVEPKSCDKTHTC
PPCPAPELLGGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKALPAP
IEKTISKAKGQPREPQVYTLPPSREEMTKNQVSLT
CLVKGFYPSDIAVEWESNGQPENNYKTTPPVLDSD
GSFFLYSKLTVDKSRWQQGNVFSCSVMHEALHNHY
TQKSLSLSPGK
SEQ ID NO: 8
MDMRVPAQLLGLLLLWFPGSRCDIQMTQSPSSVSA
SVGDRVTITCRASQGISGWLAWYQQKPGKAPKFLI
YAASTLQSGVPSRFSGSGSGTDFTLTISSLQPEDF
ATYYCQQANSFPPTFGGGTKVEIKRTVAAPSVFIF
PPSDEQLKSGTASVVCLLNNFYPREAKVQWKVDNA
LQSGNSQESVTEQDSKDSTYSLSSTLTLSKADYEK
HKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dithel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-191P4D12 antibody or antigen binding fragment thereof comprises a heavy chain variable region comprising complementarity determining regions (CDRs) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 4 below.

TABLE 4

| | VH of SEQ ID NO: 22 | VL of SEQ ID NO: 23 |
|---|---|---|
| CDR1 | SYNMN | RASQGISGWLA |
| | (SEQ ID NO:9) | (SEQ ID NO: 12) |
| CDR2 | YISSSSSTIYYADSVKG | AASTLQS |
| | (SEQ ID NO: 10) | (SEQ ID NO: 13) |
| CDR3 | AYYYGMDV | QQANSFPPT |
| | (SEQ ID NO: 11) | (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 5 below.

TABLE 5

| | VH of SEQ ID NO: 22 | VL of SEQ ID NO: 23 |
|---|---|---|
| CDR1 | GFTFSSYN | QGISGW |
| | (SEQ ID NO: 16) | (SEQ ID NO: 19) |
| CDR2 | ISSSSSTI | AAS |
| | (SEQ ID NO: 17) | (SEQ ID NO: 20) |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR H1 comprising an amino acid sequence of SEQ ID NO:9, CDR H2 comprising an amino acid sequence of SEQ ID NO:10, CDR H3 comprising an amino acid sequence of SEQ ID NO:11, CDR L1 comprising an amino acid sequence of SEQ ID NO:NO:12, CDR L2 comprising an amino acid sequence of SEQ ID NO:NO:13, and CDR L3 comprising an amino acid sequence of SEQ ID NO:NO:14.

In some embodiments, the antibody or antigen binding fragment thereof comprises CDR H1 comprising an amino acid sequence of SEQ ID NO:16, CDR H2 comprising an amino acid sequence of SEQ ID NO:17, CDR H3 comprising an amino acid sequence of SEQ ID NO:18, CDR L1 comprising an amino acid sequence of SEQ ID NO:NO:19, CDR L2 comprising an amino acid sequence of SEQ ID NO:NO:20, and CDR L3 comprising an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also may be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C- terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7.

In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8.

In some embodiments, the anti-191P4D12 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-191P4D12 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:

(a) the heavy chain variable region comprises CDRs comprising the amino acid sequences of the heavy chain variable region CDRs set forth in the antibody produced by a hybridoma deposited under the American Type Culture Collection (ATCC) Accession NO: PTA-11267;

(b) the light chain variable region comprises CDRs comprising the amino acid sequences of the light chain variable region CDRs set forth in the antibody produced by a hybridoma deposited under the American Type Culture Collection (ATCC) Accession NO: PTA-11267.

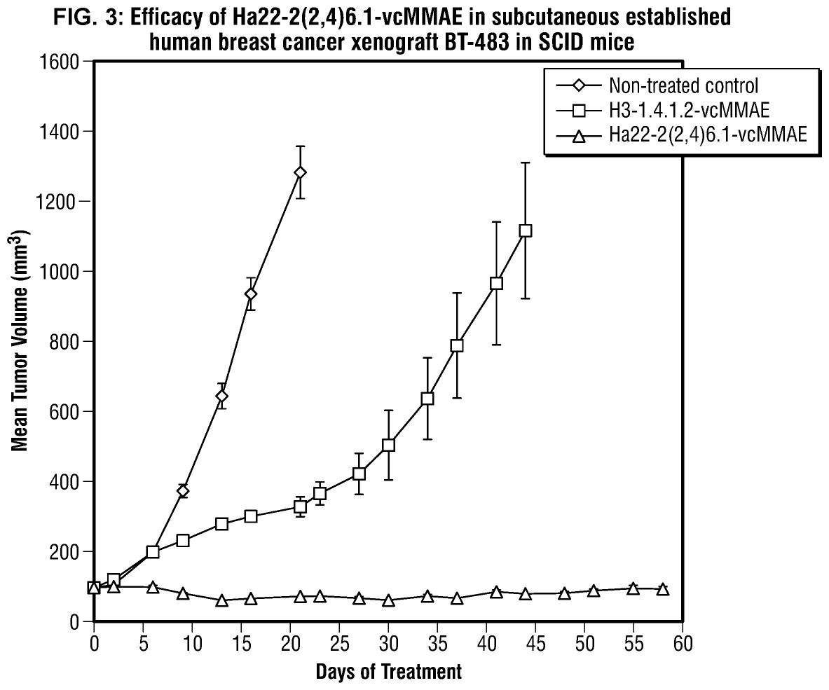

In some embodiments, the anti-191P4D12 antibody provided herein comprises heavy and light chain variable regions of an antibody designated Ha22-2(2,4)6.1 produced by a hybridoma deposited under the American Type Culture Collection (ATCC) Accession NO: PTA-11267 (See, FIG. 3), or heavy and light variable regions comprising amino acid sequences that are homologous to the amino acid sequences of the heavy and light chain variable regions of Ha22-2(2,4)6.1, and wherein the antibodies retain the desired functional properties of the anti-191P4D12 antibody provided herein. As the constant region of the antibody of the invention, any subclass of constant region can be chosen. In one embodiment, human IgG1 constant region as the heavy chain constant region and human Ig kappa constant region as the light chain constant region can be used.

In some embodiments, the anti-191P4D12 antibody provided herein comprises heavy and light chains of an antibody designated Ha22-2(2,4)6.1 produced by a hybridoma deposited under the American Type Culture Collection (ATCC) Accession NO: PTA-11267 (See, FIG. 3), or heavy and light chains comprising amino acid sequences that are homologous to the amino acid sequences of the heavy and light chains of Ha22-2(2,4)6.1, and wherein the antibodies retain the desired functional properties of the anti-191P4D12 antibody provided herein.

In some embodiments, the antibody or antigen binding fragment thereof provided herein comprises a heavy chain variable region and a light chain variable region, wherein:

(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and

(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region may be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267.

In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region may be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267.

In other embodiments, the antibody or antigen binding fragment thereof provided herein comprises a heavy chain and a light chain, wherein:

(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and

(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain may be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267.

In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain may be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2—CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, Va. 20108 on 18 Aug. 2010 and assigned Accession number PTA-11267.

5.3.2 Cytotoxic Agents (Drug Units)

In some embodiments, the ADC comprises an antibody or antigen binding fragment thereof conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug unit may be attached to the antibody through the N (amino) terminus or the C (carboxyl) terminus of the peptidic drug unit (WO 02/088172).

Exemplary auristatin embodiments include the N-terminus linked monomethylauristatin drug units DE and DF, disclosed in “Senter et al, Proceedings of the American Association for Cancer Research, Volume 45, Abstract Number 623, presented Mar. 28, 2004 and described in United States Patent Publication No. 2005/0238649, the disclosure of which is expressly incorporated by reference in its entirety.

In some embodiments, the auristatin is MMAE (wherein the wavy line indicates the covalent attachment to a linker of an antibody drug conjugate).

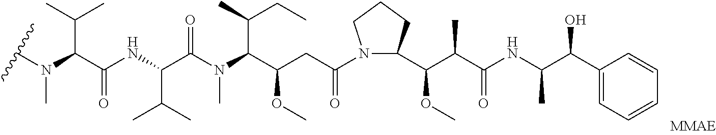 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody and p ranges from 1 to 12):

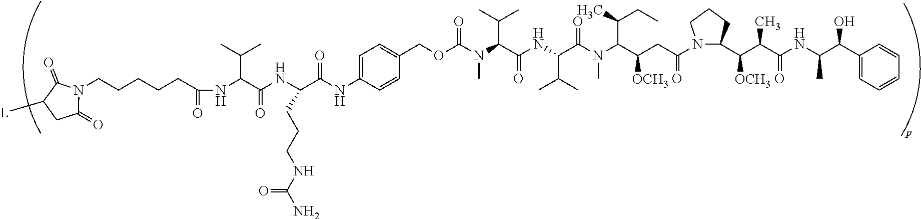

Typically, peptide-based drug units can be prepared by forming a peptide bond between two or more amino acids and/or peptide fragments. Such peptide bonds can be prepared, for example, according to the liquid phase synthesis method (see E. Schroder and K. Lake, “The Peptides”, volume 1, pp 76-136, 1965, Academic Press) that is well-known in the field of peptide chemistry. The auristatin/dolastatin drug units may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 191P4D12-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody drug conjugate, are cleaved when the antibody drug conjugate presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In other non-mutually exclusive embodiments, the linker promotes cellular internalization. In certain embodiments, the linker promotes cellular internalization when conjugated to the therapeutic agent (i.e., in the milieu of the linker-therapeutic agent moiety of the antibody-drug conjugate compound as described herein). In yet other embodiments, the linker promotes cellular internalization when conjugated to both the auristatin compound and the anti-191P4D12 antibody or antigen binding fragment thereof.

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-

wherein:-A- is a stretcher unit,

a is 0 or 1,

each -W- is independently an amino acid unit,

w is an integer ranging from 0 to 12,

-Y- is a self-immolative spacer unit, and

y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0.

5.3.3.1 Stretcher Unit

The stretcher unit (A), when present, is capable of linking an antibody unit to an amino acid unit (-W-), if present, to a spacer unit (-Y-), if present; or to a drug unit (-D). Useful functional groups that can be present on an anti-191P4D12 antibody or an antigen binding fragment thereof (e.g. Ha22-2(2,4)6.1), either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-191P4D12 antibody or an antigen binding fragment thereof. In another embodiment, sulfhydryl groups can be generated by reaction of an amino group of a lysine moiety of an anti-191P4D12 antibody or an antigen binding fragment with 2-iminothiolane (Traut's reagent) or other sulfhydryl generating reagents. In certain embodiments, the anti-191P4D12 antibody or antigen binding fragment thereof is a recombinant antibody and is engineered to carry one or more lysines. In certain other embodiments, the recombinant anti-191P4D12 antibody is engineered to carry additional sulfhydryl groups, e.g., additional cysteines.

In one embodiment, the stretcher unit forms a bond with a sulfur atom of the antibody unit. The sulfur atom can be derived from a sulfhydryl group of an antibody. Representative stretcher units of this embodiment are depicted within the square brackets of Formulas Ma and Mb below, wherein L-, -W-, -Y-, -D, w and y are as defined above, and R¹⁷ is selected from —C₁-C₁₀-C₁-C₁₀ Calkenylene-, alkynylene-, carbocyclo-, —O—(C₁-C₈ alkylene)-, 0-(C₁-C₈ alkenylene)-, —O—(C₁-C₈ alkynylene)-, -arylene-, alkylene-arylene-, —C₂-C₁₀ alkenylene-arylene, —C₂-C₁₀ alkynylene-arylene, -arylene—C₁-C₁₀ Calkylene-, -arylene—C₂-C₁₀ alkenylene-, -arylene—C₂-C₁₀ alkynylene-, —C₁-C₁₀ Calkylene-(carbocyclo)-, —C₂-C₁₀ alkenylene-(carbocyclo)-, —C₂-C₁₀ alkynylene-(carbocyclo)-, -(carbocyclo)—C₁-C₁₀ Calkylene-, -(carbocyclo)—C₂-C₁₀ alkenylene-, -(carbocyclo)—C₂-C₁₀ alkynylene, -heterocyclo-, alkylene-(heterocyclo)-, —C₂-C₁₀ alkenylene-(heterocyclo)-, —C₂-C₁₀ alkynylene-(heterocyclo)-, -(heterocyclo)—C₁-C₁₀ Calkylene-, -(heterocyclo)—C₂-C₁₀ alkenylene-, -(heterocyclo)—C₁-C₁₀ Calkynylene-, —(CH₂CH₂O)r-, or —(CH₂CH₂O)r-CH₂—, and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted.

In some embodiments, R¹⁷ is selected from —C₁-C₁₀ Calkylene-, -carbocyclo-, —O—(C₁-C₈ alkylene)-, -arylene-, —C₁-C₁₀ Calkylene-arylene-, -arylene—C₁-C₁₀ Calkylene-, alkylene-(carbocyclo)-, -(carbocyclo)—C₁-C₁₀ Calkylene-, —C₃-C₈ heterocyclo-, —C₁-C₁₀ Calkylene-(heterocyclo)-, -(heterocyclo)—C₁-C₁₀ Calkylene-, —(CH₂CH₂O)r-, and —(CH₂CH₂O)r-CH₂—; and r is an integer ranging from 1-10, wherein said alkylene groups are unsubstituted and the remainder of the groups are optionally substituted.

It is to be understood from all the exemplary embodiments that even where not denoted expressly, 1 to 20 drug units can be linked to an antibody unit (p=1-20).

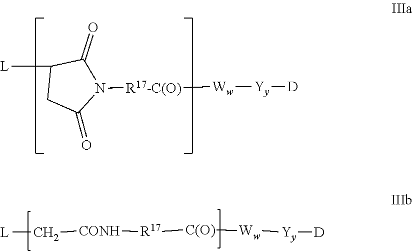

An illustrative stretcher unit is that of Formula Ma wherein R¹⁷ is —(CH₂)5-:

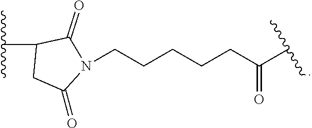 [SMILES: *C(=O)CCCCCN1C(=O)CC(*)C1=O]

Another illustrative stretcher unit is that of Formula Ma wherein R¹⁷ is —(CH₂CH₂O)r-CH 2—; and r is 2:

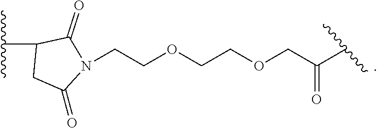 [SMILES: *C(=O)COCCOCCN1C(=O)CC(*)C1=O]

An illustrative Stretcher unit is that of Formula Ma wherein R¹⁷ is arylene- or arylene—C₁-C₁₀ Calkylene-. In some embodiments, the aryl group is an unsubstituted phenyl group.

Still another illustrative stretcher unit is that of Formula Mb wherein R¹⁷ is —(CH₂)5-:

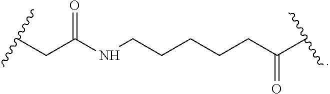 [SMILES: *CC(=O)NCCCCCC(*)=O]

In certain embodiments, the stretcher unit is linked to the antibody unit via a disulfide bond between a sulfur atom of the antibody unit and a sulfur atom of the stretcher unit. A representative stretcher unit of this embodiment is depicted within the square brackets of Formula IV, wherein R¹⁷, L-, -W-, -Y-, -D, w and y are as defined above.

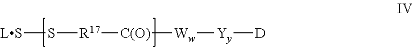

It should be noted that throughout this application, the S moiety in the formula below refers to a sulfur atom of the antibody unit, unless otherwise indicated by context.

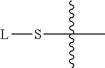

In certain of the structural descriptions of sulfur linked ADC herein the antibody is represented as “L”. It could also be indicated as “Ab-S”. The inclusion of “S” merely indicated the sulfur-linkage feature, and does not indicate that a particular sulfur atom bears multiple linker-drug moieties. The left parentheses of the structures using the “Ab-S” description may also be placed to the left of the sulfur atom, between Ab and S, which would be an equivalent description of the ADC of the invention described throughout herein.

In yet other embodiments, the stretcher contains a reactive site that can form a bond with a primary or secondary amino group of an antibody unit. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas Va and Vb, wherein —R¹⁷—, L-, -W-, -Y-, -D, w and y are as defined above;

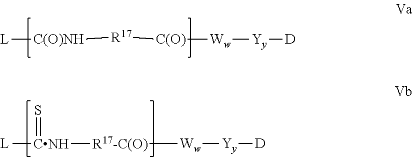

In some embodiments, the stretcher contains a reactive site that is reactive to a modified carbohydrate's (—CHO) group that can be present on an antibody unit. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative stretcher units of this embodiment are depicted within the square brackets of Formulas VIa, VIb, and VIc, wherein -R¹⁷—, L-, -W-, -Y-, -D, w and y are as defined as above.

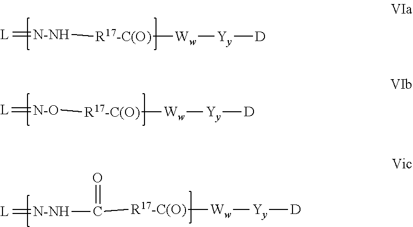

5.3.3.2 Amino Acid Unit

The amino acid unit (-W-), when present, links the stretcher unit to the spacer unit if the spacer unit is present, links the stretcher unit to the drug unit if the spacer unit is absent, and links the antibody unit to the drug unit if the stretcher unit and spacer unit are absent.

W_{w}- can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each -W- unit independently has the formula denoted below in the square brackets, and w is an integer ranging from 0 to 12:

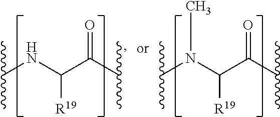

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH2, —CH₂COOH, —CH₂CH₂CONH2, —CH₂CH₂COOH, —(CH₂)₃NHC(═NH)NH₂, —(CH₂)₃NH₂, —(CH₂)₃NHCOCH3, —(CH₂)₃NHCHO, —(CH₂)₄NHC(═NH)NH₂, —(CH₂)₄NH₂, —(CH₂)₄NHCOCH3, —(CH₂)₄NHCHO, —(CH₂)₃NHCONH2, —(CH₂)₄NHCONH2, —CH₂CH₂CH(OH)CH₂NH₂, 2-pyridylmethyl-, 3-pyridylmethyl-, 4-pyridylmethyl-, phenyl, cyclohexyl,

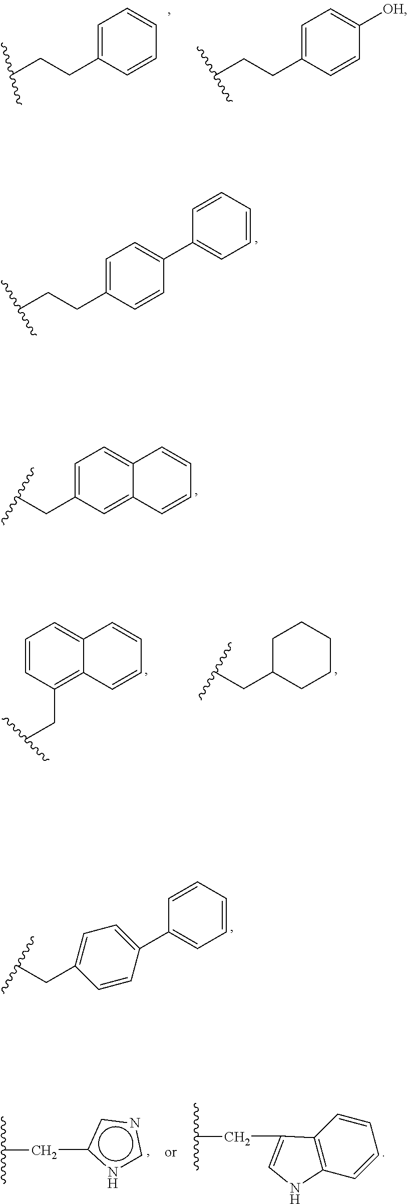

In some embodiments, the amino acid unit can be enzymatically cleaved by one or more enzymes, including a cancer or tumor-associated protease, to liberate the drug unit (-D), which in one embodiment is protonated in vivo upon release to provide a drug (D).

In certain embodiments, the amino acid unit comprises natural amino acids. In other embodiments, the amino acid unit comprises non-natural amino acids. Illustrative W_{w} units are represented by Formulas VII-IX below:

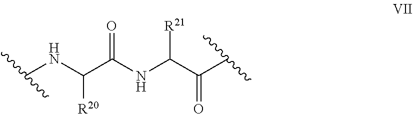

wherein R²⁰ and R²¹ are as follows:

| R²⁰ | R²¹ |
|---|---|
| Benzyl | (CH₂)₄NH₂; |
| Methyl | (CH₂)₄NH₂; |
| Isopropyl | (CH₂)₄NH₂; |
| Isopropyl | (CH₂)₃NHCONH₂; |
| Benzyl | (CH₂)₃NHCONH₂; |
| Isobutyl | (CH₂)₃NHCONH₂; |
| sec-butyl | (CH₂)₃NHCONH₂; |
| 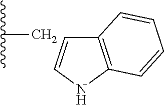 | (CH₂)₃NHCONH₂; |
| Benzyl | methyl; |
| Benzyl | (CH₂)₃NHC(═NH)NH₂; |

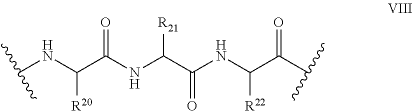

wherein R²⁰, R²¹ and R²² are as follows:

| R²⁰ | R²¹ | R²² |
|---|---|---|
| Benzyl | benzyl | (CH₂)₄NH₂; |
| Isopropyl | benzyl | (CH₂)₄NH₂; and |
| H | benzyl | (CH₂)₄NH₂; |

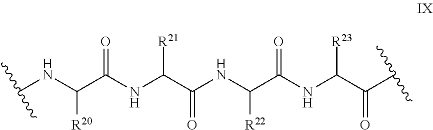

wherein R²⁰, R²¹, R²² and R²³ are as follows:

| R²⁰ | R²¹ | R²² | R²³ |
|---|---|---|---|
| H | benzyl | isobutyl | H; and |
| Methyl | isobutyl | methyl | isobutyl. |

Exemplary amino acid units include, but are not limited to, units of Formula VII above where: R²⁰ is benzyl and R²¹ is —(CH₂)₄NH₂; R²⁰ is isopropyl and R²¹ is —(CH₂)₄NH₂; or R²⁰ is isopropyl and R²¹ is —(CH₂)₃NHCONH2.

Another exemplary amino acid unit is a unit of Formula VIII wherein R²⁰ is benzyl, R²¹ is benzyl, and R²² is —(CH₂)₄NH₂.

Useful -W_{w}- units can be designed and optimized in their selectivity for enzymatic cleavage by a particular enzyme, for example, a tumor-associated protease. In one embodiment, a -W_{w}-unit is that whose cleavage is catalyzed by cathepsin B, C and D, or a plasmin protease.

In one embodiment, -W_{w}- is a dipeptide, tripeptide, tetrapeptide or pentapeptide. When R¹⁹, R²⁰, R²¹, R²²or R²³ is other than hydrogen, the carbon atom to which R¹⁹, R²⁰, R²¹, R²² or R²³ is attached is chiral.

Each carbon atom to which R¹⁹, R²⁰, R²¹, R²²or R²³ is attached is independently in the (S) or (R) configuration.

In one specific embodiment, the amino acid unit is valine-citrulline (vc or Val-Cit). In another specific embodiment, the amino acid unit is phenylalanine-lysine (i.e., fk). In yet another specific embodiment, the amino acid unit is N-methylvaline-citrulline. In yet another specific embodiment, the amino acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

5.3.3.3 Spacer Unit

The spacer unit (-Y-), when present, links an amino acid unit to the drug unit when an amino acid unit is present. Alternately, the spacer unit links the stretcher unit to the drug unit when the amino acid unit is absent. The spacer unit also links the drug unit to the antibody unit when both the amino acid unit and stretcher unit are absent.

Spacer units are of two general types: non self-immolative or self-immolative. A non self-immolative spacer unit is one in which part or all of the spacer unit remains bound to the drug unit after cleavage, particularly enzymatic, of an amino acid unit from the antibody drug conjugate. Examples of a non self-immolative spacer unit include, but are not limited to a (glycine-glycine) spacer unit and a glycine spacer unit (both depicted in Scheme 1) (infra). When a conjugate containing a glycine-glycine spacer unit or a glycine Spacer unit undergoes enzymatic cleavage via an enzyme (e.g., a tumor-cell associated-protease, a cancer-cell-associated protease or a lymphocyte-associated protease), a glycine-glycine-drug unit or a glycine-drug unit is cleaved from L-Aa-Ww-. In one embodiment, an independent hydrolysis reaction takes place within the target cell, cleaving the glycine-drug unit bond and liberating the drug.

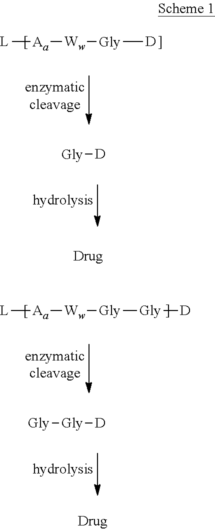

In some embodiments, a non self-immolative spacer unit (-Y-) is -Gly-. In some embodiments, a non self-immolative spacer unit (-Y-) is -Gly-Gly-.

In one embodiment, the spacer unit is absent (-Y_{y}-where y=0).

Alternatively, an antibody drug conjugate containing a self-immolative spacer unit can release -D. As used herein, the term “self-immolative spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, -Y_{y}- is a p-aminobenzyl alcohol (PAB) unit (see Schemes 2 and 3) whose phenylene portion is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, - nitro or -cyano; and m is an integer ranging from 0-4. The alkyl, alkenyl and alkynyl groups, whether alone or as part of another group, can be optionally substituted.

In some embodiments, -Y- is a PAB group that is linked to -W_{w}-via the amino nitrogen atom of the PAB group, and connected directly to -D via a carbonate, carbamate or ether group. Without being bound by any particular theory or mechanism, Scheme 2 depicts a possible mechanism of Drug release of a PAB group which is attached directly to -D via a carbamate or carbonate group as described by Toki et al., 2002, J Org. Chem. 67:1866-1872.

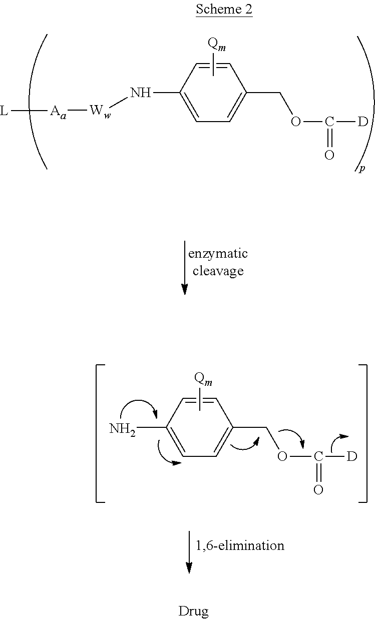

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Without being bound by any particular theory or mechanism, Scheme 3 depicts a possible mechanism of drug release of a PAB group which is attached directly to -D via an ether or amine linkage, wherein D includes the oxygen or nitrogen group that is part of the drug unit.

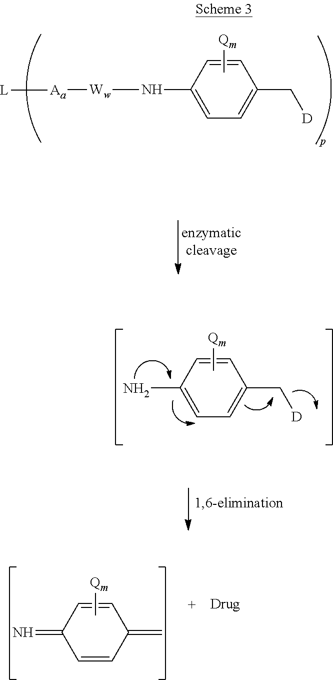

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J Amer. Chem. Soc. 94:5815) and 2-aminophenylpropionic acid amides (Amsberry et al., 1990, J Org. Chem. 55:5867). Elimination of amine-containing drugs that are substituted at the α-position of glycine (Kingsbury et al., 1984, 1 Med Chem. 27:1447) are also examples of self-immolative spacers.

In one embodiment, the spacer unit is a branched bis(hydroxymethyl)-styrene (BHMS) unit as depicted in Scheme 4, which can be used to incorporate and release multiple drugs.

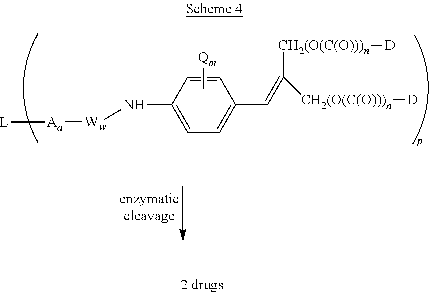

In Scheme 4, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4;
n is 0 or 1; and p ranges ranging from 1 to about 20. The alkyl, alkenyl and alkynyl groups, whether alone or as part of another group, can be optionally substituted.

In some embodiments, the -D units are the same. In yet another embodiment, the -D moieties are different.

In one aspect, spacer units (-Y_{y}—) are represented by Formulas X-XII:

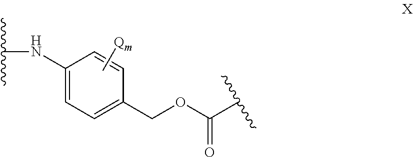

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

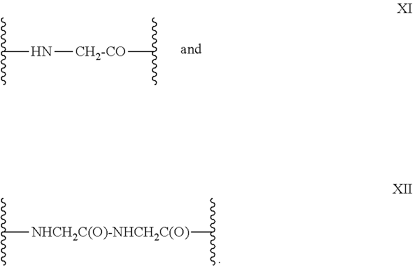

Embodiments of the Formula I and II comprising antibody-drug conjugate compounds can include:

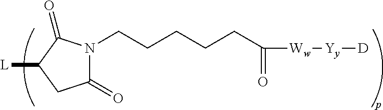

wherein w and y are each 0, 1 or 2, and,

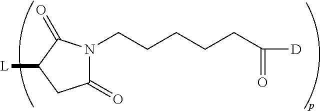

wherein w and y are each 0,

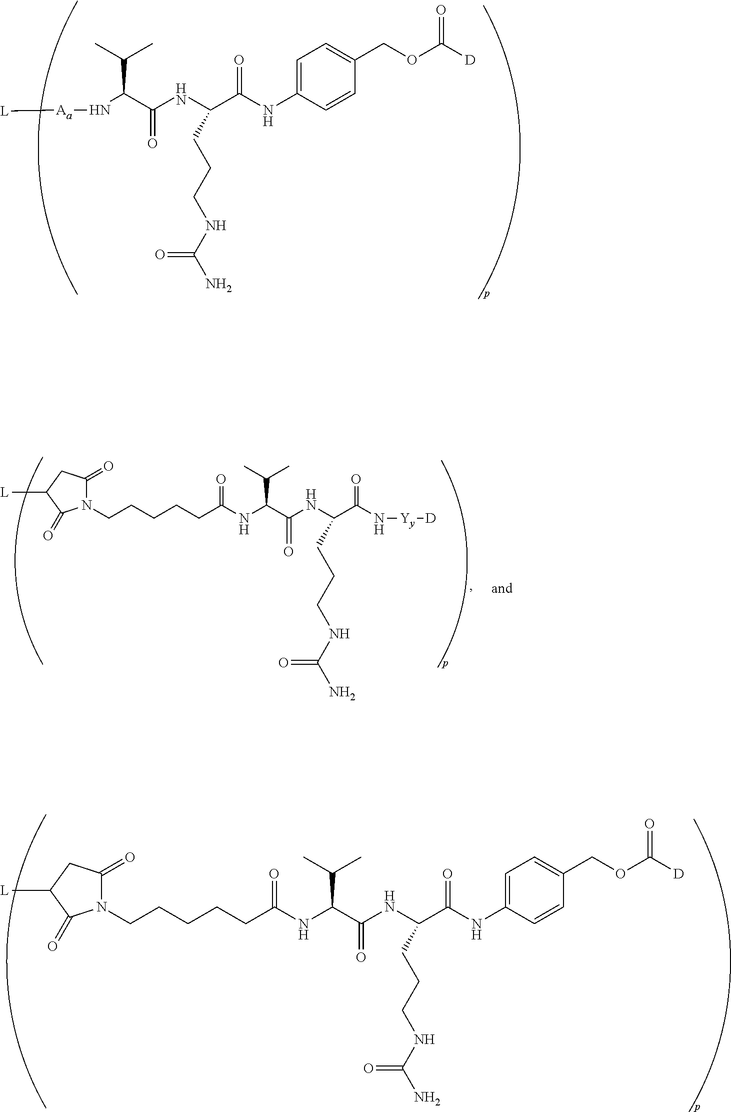

5.3.3.4 Drug Loading

Drug loading is represented by p and is the average number of drug units per antibody in a molecule. Drug loading may range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions may be characterized by conventional means such as mass spectroscopy and, ELISA assay. The quantitative distribution of ADC in terms of p may also be determined. In some instances, separation, purification, and characterization of homogeneous ADC where p is a certain value from ADC with other drug loadings may be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In certain embodiments, fewer than the theoretical maximum of drug units are conjugated to an antibody during a conjugation reaction. An antibody may contain, for example, lysine residues that do not react with the drug-linker intermediate or linker reagent. Generally, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH₂, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S.C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

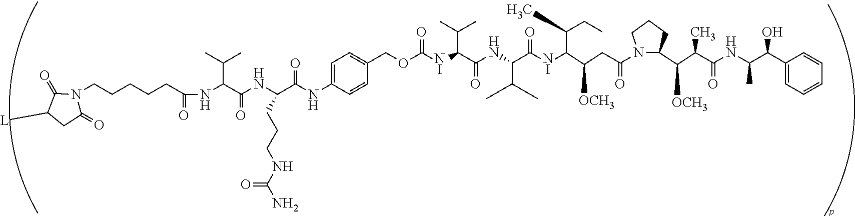

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1K) antibody conjugated to the microtubule-disrupting agent (MMAE) via a protease-cleavable linker (Challita-Eid PM et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco JA et al, Blood. 2003 Aug. 15;102(4):1458-65).

As described above and in in U.S. Pat. No. 8,637,642, AGS-22M6E is an ADC derived from a murine hybridoma cell line. Enfortumab vedotin is the a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions may be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs may further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds may be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 below.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM. In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises

(a) an antibody drug conjugate comprising the following structure:

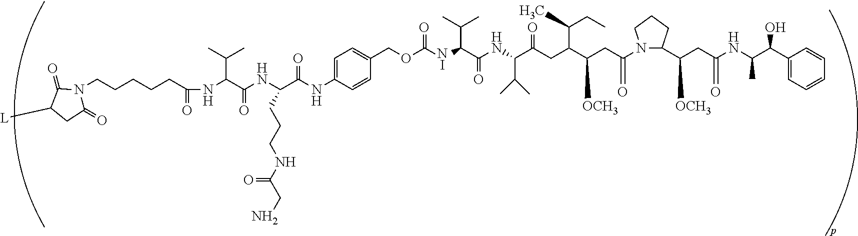

wherein L- represents the antibody or antigen binding fragment thereof and p is from 1 to10; and (b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:

(a) an antibody drug conjugate comprising the following structure:

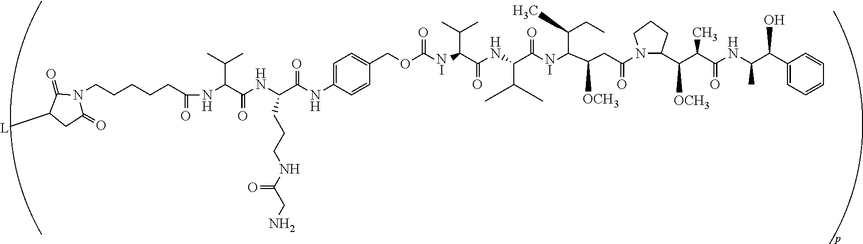

wherein L- represents the antibody or antigen binding fragment thereof and p is from 1 to10; and (b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:

(a) an antibody drug conjugate comprising the following structure:

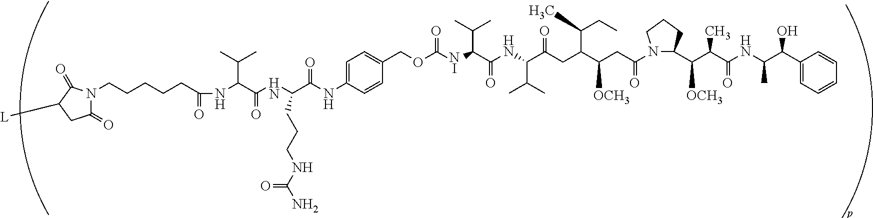

wherein L- represents the antibody or antigen binding fragment thereof and p is from 1 to10; and (b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated. Other exemplary pharmaceutical compositions are provided in the Experimental section below.

A primary solvent in a vehicle may be either aqueous or non-aqueous in nature. In addition, the vehicle may contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein may contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions may be in the form of a sterile injectable aqueous or oleagenous suspension. This suspension may be formulated using suitable dispersing or wetting agents and suspending agents disclosed herein or known to the skilled artisan. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl-and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In one embodiment, the pharmaceutical compositions provided herein may be formulated for single or multiple dosage administration. The single dosage formulations are packaged in an ampoule, a vial, or a syringe. The multiple dosage parenteral formulations may contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein may be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein may be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They may also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

5.5 Methods of Using the Pharmaceutical Compositions in a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the chemotherapeutic agent is administered separately. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation, used in combination with the pharmaceutical composition provided herein, can be either external or internal to the patient being treated. When the source is external to the patient, the therapy is known as external beam radiation therapy (EBRT). When the source of radiation is internal to the patient, the treatment is called brachytherapy (BT).

The above described therapeutic regimens may be further combined with additional cancer treating agents and/or regimes, for example additional chemotherapy, cancer vaccines, signal transduction inhibitors, agents useful in treating abnormal cell growth or cancer, antibodies (e.g. Anti-CTLA-4 antibodies as described in WO/2005/092380 (Pfizer)) or other ligands that inhibit tumor growth by binding to IGF-1R, and cytokines.

When the mammal is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3- ‘-(trifluoromethyl)propionanilide) may be used.

In some embodiments, the pharmaceutical provided herein in used in combination with a second therapeutic agent, e.g., for treating a cancer.

In some embodiments, the second therapeutic agent is an immune checkpoint inhibitor. As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhlL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second active agents as described herein where appropriate for treating diseases described herein and understood in the art.

In some embodiments, the checkpoint inhibitor is administered prior to the administration of the present pharmaceutical composition. In other embodiments, the checkpoint inhibitor is administered simultaneously (e.g., in the same dosing period) with the pharmaceutical composition provided herein. In yet other embodiments, the checkpoint inhibitor is administered after the administration of the pharmaceutical composition provided herein.

In some embodiments, the amount of the checkpoint inhibitor can be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, or at least 3 mg, such as at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, or at least 1 mg/ml, and such as at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

5.6 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses may be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses may be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein may be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial cancer who have shown disease progression or relapse during or after treatment with an immune checkpoint inhibitor. In some embodiments, the antibody drug conjugate is administered to patients with metastatic urothelial cancer who have shown disease progression or relapse during or after treatment with an immune checkpoint inhibitor.

In other more specific embodiments, the antibody drug conjugate formulated in the pharmaceutical composition provided herein is administered at a dose of about about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial cancer who have shown disease progression or relapse during or after treatment with an immune checkpoint inhibitor. In some embodiments, the antibody drug conjugate is administered to patients with metastatic urothelial cancer who have shown disease progression or relapse during or after treatment with an immune checkpoint inhibitor.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1— Ha22-2(2,4)6.1vcMMAE Inhibit Growth of Tumors In Vivo

The significant expression of 191P4D12 on the cell surface of tumor tissues, together with its restrictive expression in normal tissues makes 191P4D12 a good target for antibody therapy and similarly, therapy via ADC. Thus, the therapeutic efficacy of Ha22-2(2,4)6.1vcMMAE in human bladder, lung, breast, and pancreatic cancer xenograft mouse models is evaluated.

Antibody drug conjugate efficacy on tumor growth and metastasis formation is studied in mouse cancer xenograft models (e.g. subcutaneous and orthotopically).

Subcutaneous (s.c.) tumors are generated by injection of 5×10⁴- 10⁶ cancer cells mixed at a 1:1 dilution with Matrigel (Collaborative Research) in the right flank of male SCID mice. To test ADC efficacy on tumor formation, ADC injections are started on the same day as tumor-cell injections. As a control, mice are injected with either purified human IgG or PBS; or a purified MAb that recognizes an irrelevant antigen not expressed in human cells. In preliminary studies, no difference is found between control IgG or PBS on tumor growth. Tumor sizes are determined by caliper measurements, and the tumor volume is calculated as width²×Length/2, wherein width is the smallest dimension and length is the largest dimension. Mice with subcutaneous tumors greater than 1.5 cm in diameter are sacrificed.

An advantage of xenograft cancer models is the ability to study neovascularization and angiogenesis. Tumor growth is partly dependent on new blood vessel development. Although the capillary system and developing blood network is of host origin, the initiation and architecture of the neovasculature is regulated by the xenograft tumor (Davidoff et al., Clin Cancer Res. (2001) 7:2870; Solesvik et al., Eur J Cancer Clin Oncol. (1984) 20:1295). The effect of antibody and small molecule on neovascularization is studied in accordance with procedures known in the art, such as by IHC analysis of tumor tissues and their surrounding microenvironment.

Ha22-2(2,4)6.1ADC inhibits formation of lung and breast cancer xenografts. These results indicate the utility of Ha22-2(2,4)6.1ADC in the treatment of local and metastatic (including malignant or metastatic malignant) stages of cancer, including for example, lung cancer and breast cancer.

191P4D12 ADCs:

Monoclonal antibodies against 191P4D12 and its conjugation to MMAE are described above. The Ha22-2(2,4)6.1vcMMAE is characterized by FACS, and other methods known in the art to determine its capacity to bind 191P4D12.

Cell Lines and Xenografts:

The BT-483 and HPAC cells are maintained in DMEM, supplemented with L-glutamine and 10% FBS, as known in the art. AG-L4 xenografts are maintained by serial propagation in SCID mice.

Efficacy of Ha22-2(2,4)6.1-vcMMAE in subcutaneous established human lung cancer xenograft AG-L4 in SCID mice

In another experiment, patient-derived lung cancer xenograft AG-L4 was maintained by serial passages in SCID mice. Stock tumors were harvested sterilely and minced into 1 mm³ pieces. Six (6) pieces were implanted into the flank of individual SCID mice. Tumors were allowed to grow untreated until they reached an approximate volume of 200 mm³. The Ha22-2(2,4)6.1vcMMAE and the control ADC were dosed at 10 mg/kg every seven (7) days for two doses by intravenous bolus injection. The amount of ADC administered was based on the individual body weight of each animal obtained immediately prior to dosing. Tumor growth was monitored using caliper measurements every 3 to 4 days. Tumor volume was calculated as Width²×Length/2, where width is the smallest dimension and length is the largest dimension.

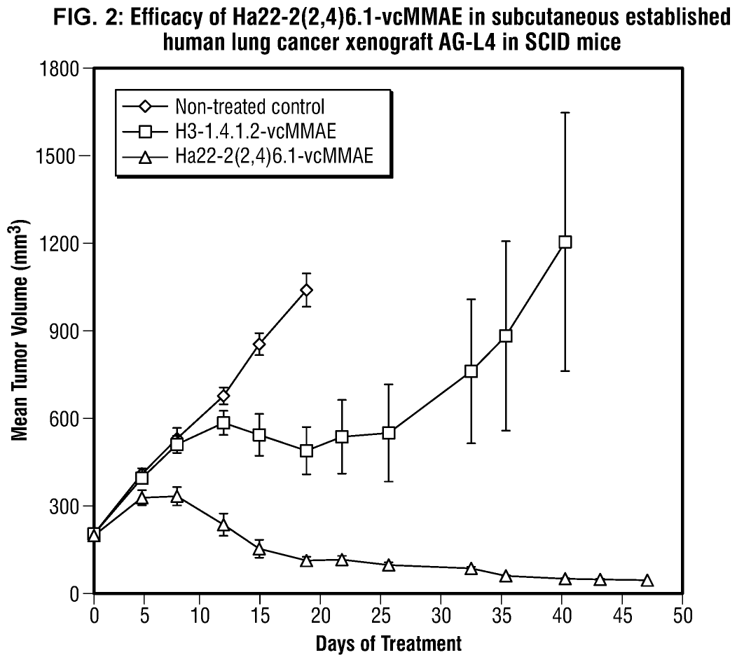

The results show that treatment with Ha22-2(2,4)6.1-vcMMAE significantly inhibited the growth of AG-L4 lung cancer xenografts implanted subcutaneously in nude mice compared to the control ADC. (FIG. 2). Additionally, other 191P4D12 MAbs were utilized in this study. The results are not shown.

Efficacy of Ha22-2(2,4)6.1-vcMMAE in subcutaneous established human breast cancer xenograft BT-483 in SCID mice

In this experiment, human breast cancer BT-483 cells were used to generate stock xenografts, which were maintained by serial passages in SCID mice. Stock tumors were harvested sterilely and minced into 1 mm³ pieces. Six (6) pieces were implanted into the flank of individual SCID mice. Tumors were allowed to grow untreated until they reached an approximate volume of 100 mm³. The Ha22-2(2,4)6.1vcMMAE and the control ADC were dosed at 5 mg/kg every four (4) days for four (4) doses by intravenous bolus injection. The amount of ADC administered was based on the individual body weight of each animal obtained immediately prior to dosing. Tumor growth was monitored using caliper measurements every 3 to 4 days. Tumor volume was calculated as Width²×Length/2, where width is the smallest dimension and length is the largest dimension.

The results show that treatment with Ha22-2(2,4)6.1-vcMMAE significantly inhibited the growth of BT-483 breast tumor xenografts implanted subcutaneously in SCID mice compared to the control ADC. (FIG. 3). Additionally, other 191P4D12 MAbs were utilized in this study. The results are not shown.

### CONCLUSION

In summary, FIGS. 2 and 3, show that the 191P4D12 ADC entitled Ha22-2(2,4)6.1vcMMAE significantly inhibited the growth of tumors cells that express 191P4D12 when compared to control ADCs. Thus, the Ha22-2(2,4)6.1vcMMAE can be used for therapeutic purposes to treat and manage various cancers, including, for example, breast cancer and lung cancer.

### 6.2 Example 2— Detection of 191P4D12 Protein in Cancer Patient Specimens by IHC

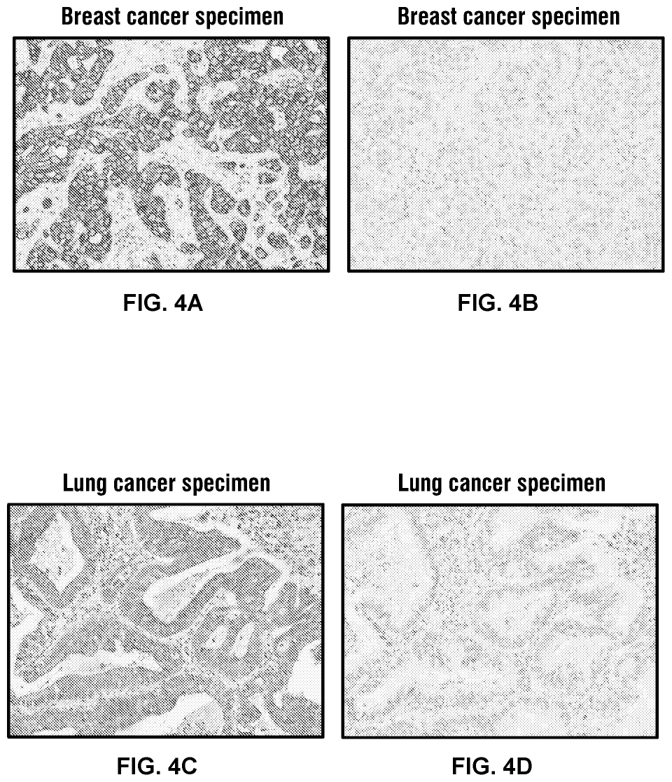

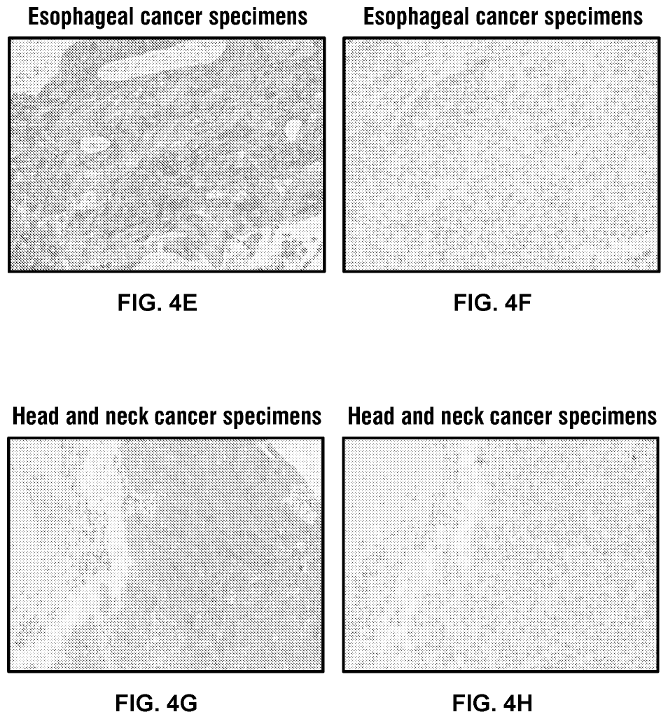

Expression of 191P4D12 protein by immunohistochemistry was tested in patient tumor specimens from (i) breast, (ii) lung, (iii) esophageal, and (iv) head and neck patients. Briefly, formalin fixed, paraffin wax-embedded tissues were cut into four (4) micron sections and mounted on glass slides. The sections were de-waxed, rehydrated and treated with EDTA antigen retrieval solution (Biogenex, San Ramon, CA) in the EZ-Retriever microwave (Biogenex, San Ramon, CA) for 30 minutes at 95° C. Sections were then treated with 3% hydrogen peroxide solution to inactivate endogenous peroxidase activity. Serum-free protein block (Dako, Carpenteria, CA) was used to inhibit non-specific binding prior to incubation with monoclonal mouse anti-191P4D12 antibody or an isotype control. Subsequently, the sections were treated with the Super Sensitive™ Polymer-horseradish peroxidase (HRP) Detection System which consists of an incubation in Super Enhancer™ reagent followed by an incubation with polymer-HRP secondary antibody conjugate (BioGenex, San Ramon, CA). The sections were then developed using the DAB kit (BioGenex, San Ramon, CA). Nuclei were stained using hematoxylin, and analyzed by bright field microscopy. Specific staining was detected in patient specimens using the 191P4D12 immunoreactive antibody, as indicated by the brown staining. (See, FIGS. 4A, 4C, 4E, and 4G). In contrast, the control antibody did not stain either patient specimen. (See, FIGS. 4B, 4D, 4F, and 4H,).

The results show expression of 191P4D12 in the tumor cells of patient bladder, breast, pancreatic, lung, ovarian, esophageal, and head and neck cancer tissues. These results indicate that 191P4D12 is expressed in human cancers and that antibodies directed to this antigen and the antibody drug conjugate designated Ha22-2(2,4)6.1vcMMAE) are useful for diagnostic and therapeutic purposes. (FIGS. 4A-H).

### 6.3 Example 3— Exemplary Cancers that can be Treated by the Methods

To select certain exemplary cancers that can be treated by the methods provided herein, the prevalence of Nectin-4 expression was determined at the RNA and protein level in cancer specimens. Briefly, the prevalence of Nectin-4 expression at the RNA level was first determined using patient sample data from the The Cancer Genome Atlas (TCGA) database. Two separate IHC methods were also used to determine Nectin-4 protein expression on tissue samples from cancer patients. As shown by Table 6, the prevalence of Nectin-4 RNA expression is similar to prevalence of Nectin-4 expression at the protein level. Different tumors identified to prevalently express both Nectin-4 RNA and Nectin-4 protein were evaluated with respect to their sensitivity to MMAE or, if data for MMAE was not available, to Vinca, which exerts cytotoxicity via the same mechanism as MMAE by blocking microtubule polymerization.

TABLE 6
Prevalence of Nectin-4 Expression by RNA Is Similar
to Nectin-4 Prevalence in IHC Across Tumor Types

| | IHC | TCGA | IHC |
|---|---|---|---|
| | METHOD 1 | RNA | METHOD 2 |
| Breast | 78% (512/654) | 93% (1036/1114) | 90% (199/220) |
| NSCLC- | 62% (145/235) | 90% (451/501) | |
| Squamous | | | |
| NSCLC- | 64% (136/212) | 88% (466/530) | |
| Adeno- | | | |
| carcinoma | | | |
| Head & Neck- | 59% (79/135) | 96% (501/522) | 77% (98/128) |
| Squamous | | | |
| Gastric (GEJ) | | 26% (108/416) | 66% (94/143) |

These results in Table 6 demonstrate that squamous NSCLC, Gastric (GEJ) cancer, HNSCC, NSCLC-adenocarcinoma, head & neck cancer-squamous, and breast cancer (including HR+/HER2− breast cancer and TNBC) prevalently expressed Nectin-4 at both RNA and protein level. These cancers were also sensitive to cytotoxicity from MMAE, Vinca, or both MMAE and Vinca, both of which exert cytotoxicity via the mechanism of blocking microtubule polymerization. Therefore, squamous NSCLC, Gastric (GEJ), HNSCC, NSCLC-adenocarcinoma, head & neck cancer-squamous, and breast cancer (including HR+/HER2− breast cancer and TNBC) can be treated by the methods provided herein and are tested in the human studies described in the Example below.

### 6.4 Example 4— an Open-Label, Multicenter, Multi-Cohort, Phase 2 Study to Evaluate Anti-191P4D12-ADC in Subjects with Previously Treated Locally Advanced or Metastatic Malignant Solid Tumors

6.4.1. Summary of the Study

6.4.1.1. SYNOPSIS

6.4.2. About the Study

6.4.2.1. ENFORTUMAB VEDOTIN AND ITS EFFICACY IN NONCLINICAL AND CLINICAL STUDIES

6.4.2.1.1. Enfortumab Vedotin

Enfortumab vedotin is an ADC comprised of a fully human immunoglobulin G1 kappa (IgG1K) antibody conjugated to the microtubule-disrupting agent (MMAE) via a protease-cleavable linker (Challita-Eid PM et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco JA et al, Blood. 2003 Aug. 15;102(4):1458-65).

6.4.2.1.2. Nonclinical and Clinical Data

Pharmacology

AGS-22M6E is an ADC derived from a murine hybridoma cell line that was used in pharmacology and toxicology studies, as well as in a completed phase I study. Enfortumab vedotin, a Chinese hamster ovary (CHO) cell line-derived AGS-22M6E ADC is the final product used for clinical development. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In in vitro pharmacology studies, AGS-22M6E inhibited cell survival in a cell line engineered to express human 191P4D12 as well as in T47D breast cancer cell line endogenously expressing human 191P4D12, whereas AGS-22M6 (unconjugated antibody) did not affect cell growth of any of the 191P4D12 expressing cell lines.

Antitumor activity of AGS-22M6E was evaluated in a panel of tumor xenograft models representing various cancer indications in which expression of 191P4D12 has been demonstrated. AGS-22M6E significantly inhibited the tumor growth in a xenograft of a patient-derived bladder cancer in a dose-dependent manner. In a xenograft of human HR+breast cancer cell line at 5 mg/kg every 4 days for 4 doses. AGS-22M6E also significantly inhibited tumor growth in xenografts of human lung adenocarcinoma cell line and a patient-derived lung adenocarcinoma. Enfortumab vedotin and AGS-22M6E showed comparable pharmacological antitumor activity in a patient-derived TNBC cell xenograft model. In this study, the percent tumor regression relative to the starting tumor size was 68% and 70% for enfortumab vedotin and AGS-22M6E at 2 mg/kg on day 21, respectively.

Toxicology

The safety profile and pharmacokinetics of AGS-22M6E and enfortumab vedotin were comparable in cynomolgus monkeys.

Skin lesions were noted in good laboratory practice-compliant toxicity studies in rats (≥5 mg/kg; 1-fold the human systemic exposure) and in monkeys (≥1 mg/kg; 0.7-fold the human systemic exposure). The skin changes were fully reversible by the end of a 6-week recovery period.

Hyperglycemia reported in the clinical studies was absent in both the rat and monkey toxicity studies and there were no histopathological findings in the pancreas of each species.

AGS-22M6E was moderately immunogenic in male and female rats as well as in cynomolgus monkeys. AGS-22M6E and enfortumab vedotin showed comparable immunogenicity in cynomolgus monkeys.

Genotoxicity studies showed that MMAE had no discernible genotoxic potential in a reverse mutation test in bacteria (Ames test) or in a L5178Y TK+/−mouse lymphoma mutation assay. MMAE did induce chromosomal aberrations in the micronucleus test in rats which is consistent with the pharmacological action of microtubule-disrupting agents.

MMAE has no absorbance in the range of 290 nm to 700 nm and the absorbance of valine-citrulline linker-MMAE (vc-MMAE) did not meet the criteria for phototoxicity testing as defined in the ICH S10 guidance. As such, enfortumab vedotin was not considered to be sufficiently photoreactive to result in direct phototoxicity.

Testicular toxicity was noted only in rats. Findings included seminiferous tubule degeneration and hypospermia in the epididymis (≥2.0 mg/kg; approximately 1-fold the human systemic exposure at the clinically recommended does). These findings were partially reversed by the end of a 24-week recovery period. Testicular toxicity was not observed in sexually immature male monkeys administered enfortumab vedotin at doses up to 6 mg/kg (6-fold the human systemic exposure at the clinically recommended dose).

6.4.2.1.3. Clinical Data

Enfortumab vedotin is currently being tested in multiple studies including phase 1, 2, and 3 studies, both as monotherapy and in combination with several anticancer therapies. Much of the safety and efficacy data summarized below are from monotherapy data and limited to phase 1 and 2 studies.

EV-101 is a phase 1 dose escalation/expansion study that enrolled patients with 191P4D12-expressing solid tumors, (eg, metastatic urothelial cancer [UC]), who progressed on ≥1 prior chemotherapy regimen, including a cohort of patients with metastatic UC who received prior anti-PD-(L)1 therapy.

Since EV-101 enrolled patients with 191P4D12 expressing solid tumors, 191P4D12 expression data was obtained for patients with several different cancer types of relevance to this study. A validated 191P4D12 IHC test (Quest Diagnostics) was used to assign a 191P4D12 H-score ranging from 0 to 300. Ninety-eight percent of NSCLC (including squamous and non-squamous) patients expressed 191P4D12 (n=50), with a median H-score of 260. All 3 breast cancer patients pre-screened for EV-101 expressed 191P4D12 with H-scores of 140, 230 and 290. Two gastric cancer patients and 1 nasopharyngeal carcinoma patient expressed 191P4D12 with H-scores of 140, 230, and 290, respectively. To supplement the 191P4D12 expression data obtained in EV-101, the 191P4D12 IHC test was performed on tissue microarrays (TMAs) of different cancer types. 191P4D12 was expressed in the majority of tissues assessed: breast (including triple negative and HR+/HER2; 91%, n=220), gastric (66%, n=143), head and neck (77%, n=128), and esophageal (88%, n=96) cancer tissue.

6.4.2.2. Study Rationale

Currently, chemotherapy is the standard of care which have yielded relatively low response rates, short duration of responses and significant toxicities. The lack of approved therapies for patients with locally advanced or metastatic cancer and the limited activity observed with second-line chemotherapy adequately demonstrate that this population has significant unmet medical need.

A phase 1 dose escalation and expansion study (EV-101) with enfortumab vedotin monotherapy in patients with metastatic urothelial cancer and other malignant solid tumors demonstrated encouraging antitumor activity in patients with metastatic urothelial cancer. In this study, tumor reduction was seen in patients with metastatic urothelial cancer. Enfortumab vedotin provided durable responses and meaningful survival results in patients after anti-PD-(L)-1 therapy in a population with a high unmet medical need. The confirmed ORR was 45% by a central review in patients who were treated with enfortumab vedotin 1.25 mg/kg. The median overall survival (OS) of 12.3 months is encouraging given the historical median OS is ≤10.3 months (Bellmunt J et al, N Eng J Med. 2017; 376(11):1015-1026).

In a pivotal phase 2 study (EV-201), patients must have had prior treatment with platinum-containing chemotherapy or be platinum-naïve and ineligible for cisplatin treatment. The results for this study demonstrated that enfortumab vedotin was the first novel therapeutic to demonstrate substantial clinical activity in patients who progressed after platinum chemotherapy and a PD-1/L1 inhibitor. The confirmed ORR was 44% in patients treated with enfortumab vedotin 1.25 mg/kg. The median OS was 11.7 months and the median duration of response was 7.6 months. These results are highly consistent with the phase 1 EV-101 trail in the same patient population.

The tumors selected for the current study (breast, lung, head and neck, gastric, and esophageal cancer) are all representative of tumors with moderate to high 191P4D12 expression, and for which there is a need to improve outcomes in the metastatic (including malignant or metastatic malignant) and treatment refractory setting. Furthermore, the supportive preclinical sensitivity to enfortumab vedotin, along with the clinical data, and evidence based on published literature provide support for the current study.

6.4.2.3. Risk Benefit Assessment

Subjects with locally advanced or metastatic cancer selected for this study have cancer that recurred or progressed following standard of care therapy and have limited treatment options. Clinical data to date support a favorable benefit-risk ratio for enfortumab vedotin in patients with urothelial cancer. In addition, nonclinical data and clinical data support the use of enfortumab vedotin in select 191P4D12 expressing tumors other than urothelial cancer.

Despite recent advances in treatment, approximately 80% of patients do not respond to PD-1/L1 inhibitors, which are the standard of care after platinum-containing therapy has failed as an initial treatment for metastatic (including malignant or metastatic malignant) disease (Alhalabi 0 et al Oncology (Williston Park). 2019; 33(1):11-8; Kim & Seo Investig Clin Urol. 2018; 59(5):285-296; National Comprehensive Cancer Network, 2017 (Non-small cell lung cancer, NCCN clinical practice guidelines in oncology (NCCN guidelines), http://www.nccn.org/professionals/physician_gls/pdf/nscl.pdf; National Comprehensive Cancer Network (Bladder Cancer (Version 3, 2019) http:///www.nccn.org/professionals/physician_gls/pdf/bladder.pdf)). These patients have few treatment options and new therapies are urgently needed.

6.4.3. Study Objective(s) and Endpoint(s)

The primary, secondary and additional objectives and endpoints for this study are listed in Table 7

TABLE 7
Study Objective(s) and Endpoint(s)

| | |
|---|---|
| Objective(s) | Endpoint(s) |

Primary

| | |
|---|---|
| To determine the antitumor activity of | Confirmed ORR (complete response [CR] + |
| enfortumab vedotin as measured by | PR) per Response Evaluation Criteria in |
| confirmed ORR | Solid Tumors (RECIST) Version 1.1 as |
| | determined by independent review facility (BICR) |

Secondary

| | |
|---|---|
| To assess the duration of response (DOR) | Confirmed ORR per RECIST Version 1.1 |
| of enfortumab vedotin | per investigator assessment |
| To assess the disease control rate (DCR) of | DOR per RECIST Version 1.1 per BICR |
| enfortumab vedotin | assessment |
| To assess progression-free survival on | DOR per RECIST Version 1.1 per |
| enfortumab vedotin (PFS) | investigator assessment |
| To assess the OS | DCR (CR + PR + stable disease [SD]) per |
| To assess the safety and tolerability of | RECIST Version 1.1 per BICR assessment |
| enfortumab vedotin | DCR per RECIST Version 1.1 per |
| | investigator assessment |
| | PFS per BICR assessment |
| | PFS per investigator assessment |
| | OS |
| | Safety variables |
| | Adverse events (AEs) |
| | Laboratory tests |
| | Vital sign measurements |
| | 12-lead electrocardiogram |
| | Eastern Cooperative Oncology Group |
| | (ECOG) performance status |

Additional

| | |
|---|---|
| To evaluate potential genomic and/or other | Genomic and/or other biomarkers that may |
| biomarkers that may correlate with | correlate with treatment outcome, including |
| treatment outcome, including 191P4D12 | 191P4D12 expression |
| expression | Selected pharmacokinetic parameters (i.e. |
| To assess the pharmacokinetics of | Cₘₐₓ [concentration at end of infusion] and |
| enfortumab vedotin and MMAE | C_{trough} [concentration at predose]) of |
| To assess the immunogenicity of | enfortumab vedotin and MMAE |
| enfortumab vedotin | Incidence of antitherapeutic antibodies |
| To evaluate the treatment effect of | (ATA) to enfortumab vedotin |
| enfortumab vedotin on quality of life | PRO per EuroQOL 5-dimensions (EQ-5D-5L) |
| (QOL) | and pain assessment |

6.4.4. Treatment and Dose

6.4.4.1. TREATMENT

This is an open-label, multicenter, multi-cohort, phase 2 study designed to assess the antitumor activity and safety of enfortumab vedotin as a single agent in adult subjects with locally advanced or metastatic malignant solid tumors. Approximately 40 subjects are enrolled into each of the following 6 cohorts.

Cohort 1: HR+/HER2− breast cancer; or

Cohort 2: TNBC; or

Cohort 3: SQNSCLC; or

Cohort 4: NSCLC; or

Cohort 5: Head and neck cancer; or

Cohort 6: Gastric or esophageal cancer

There is 1 planned interim analysis to assess the antitumor activity for each cohort. The interim analysis is performed for a given cohort after 20 subjects with evaluable tumor response data have been treated. A Bayesian optimal design for phase 2 (BOP2) (Zhou H et al, Stat Med. 2017; 36(21):3302-3314) is used to guide interim decision rule. For each cohort, based on the 2-stage BOP2 design, when the number of subjects with confirmed response (CR and PR) is less than the prespecified minimum number of responders at stage 1, the enrollment of the cohort may stop; otherwise, the enrollment continues until the planned size of the cohort is reached.

This study consists of 3 periods: screening/baseline, treatment and follow-up.

Screening/baseline period takes place up to 28 days prior to the first dose of study treatment. In the treatment period, starting at cycle 1, subjects receive enfortumab vedotin on days 1, 8, and 15 every 28-day cycle until one of the treatment discontinuation criteria are met. Disease assessment is performed at screening/baseline and repeated every 8 weeks (56 days ±7 days) from the first dose of study treatment throughout the study until the subject has radiologically-confirmed disease progression, initiates a new subsequent anticancer therapy, dies, withdraws consent, is lost to follow-up or the study closes, whichever occurs first.

An end of treatment (EOT) visit is performed within 7 days after the last dose of enfortumab vedotin or the decision to discontinue treatment, or prior to initiation of another anticancer therapy, whichever occurs earlier. This is followed by a 30-day safety follow-up to be completed 30 days (+7 day window) from the last dose of enfortumab vedotin or the decision to discontinue treatment, in which a telephone contact with the subject is sufficient unless any assessment must be repeated to confirm resolution of drug-related AEs.

Subjects who discontinue study treatment for reasons other than radiologically-confirmed disease progression by RECIST Version 1.1 enter into a post treatment follow-up period and continue to receive imaging scans every 8 weeks (56 days ±7 days) until the subject has radiologically-confirmed disease progression, initiates a new anticancer therapy, dies, withdraws consent, is lost to follow-up or the study closes, whichever occurs first.

After 1 year on study treatment, the frequency of disease assessment is reduced to every 12 weeks (84 days ±7 days).

After radiologically-confirmed disease progression or initiation of subsequent anticancer therapy, whichever occurs first, subjects are contacted every 12 weeks in the long-term follow-up period for survival status until death, withdrawal of consent, lost to follow-up or study closure, whichever occurs first.

Confirmed ORR is the primary endpoint. Confirmed ORR is defined as the proportion of subjects whose objective response is confirmed CR or PR according to RECIST Version 1.1. Response (CR or PR) must be confirmed with a repeat imaging scan 4 weeks (28 days+7-day window) after first response.

Blood samples for pharmacokinetics and ATA are collected at protocol-specified time points. Validated assays are used to measure the concentrations of enfortumab vedotin and MMAE in serum or plasma and to assess ATA. Samples for biomarkers are collected at protocol-specified time points.

6.4.4.2. Dose

In each cohort, subjects receive enfortumab vedotin at a dose of 1.25 mg/kg as an intravenous (IV) infusion on days 1, 8 and 15 of each 28-day cycle.

6.4.5. Patient Population

The patient population consist of subjects with previously treated locally advanced or metastatic malignant solid tumors, including:

HR+/HER2− breast cancer; or

TNBC; or

SQNSCLC; or

NSCLC; or

Head and neck cancer; or

Gastric or esophageal cancer

All screening assessments must be completed and reviewed by the investigator to confirm the potential subject meets all eligibility criteria. Prospective approval of protocol deviations to eligibility criteria (also known as protocol waivers or exemptions) is not granted.

6.4.5.1. Inclusion Criteria

Subject is eligible for participation in the study if all of the following apply:

For all subjects in cohorts 1 to 6:

Institutional Review Board (IRB)/Independent Ethics Committee (IEC) approved written informed consent and privacy language as per national regulations (e.g., Health Insurance Portability and Accountability Act Authorization for US study sites) must be obtained from the subject prior to any study-related procedures (including withdrawal of prohibited medication, if applicable).

Subject is considered an adult according to local regulation at the time of signing the informed consent form (ICF).

Subject has measurable disease by RECIST version 1.1.

Subject has accessible archival tumor tissue from primary tumor or metastatic site, for which source and availability have been confirmed prior to study treatment. If no archival tumor tissue is available, the subject will have a biopsy to obtain tumor tissue prior to study treatment. If the subject is unable to undergo a biopsy due to safety concerns, enrollment into the study must be discussed with the medical monitor.

Subject has ECOG performance status of 0 or 1.

Subject has the following baseline laboratory data:

absolute neutrophil count (ANC)≥1.0×10⁹/L

platelet count ≥100×10⁹/L

hemoglobin ≥9 g/dL

serum total bilirubin ≤1.5×upper limit of normal (ULN) or ≤3×ULN for subjects with Gilbert's disease

creatinine clearance (CrCl)≥30 mL/min as estimated per institutional standards or as measured by 24-hour urine collection (glomerular filtration rate [GFR] can also be used instead of CrCl).

alanine aminotransferase (ALT) and aspartate aminotransferase (AST)≤3×ULN

Female subject is not pregnant and at least 1 of the following conditions apply:

Not a woman of childbearing potential (WOCBP)

WOCBP who agrees to follow the contraceptive guidance from the time of informed consent through at least 6 months after the last dose of study treatment administration

Female subject must agree not to breastfeed starting at screening and throughout the study period and for 6 months after the last dose of study treatment administration.

Female subject must not donate ova starting at first dose of study treatment and throughout the study period and for 6 months after the last dose of study treatment administration.

Male subject with female partner(s) of childbearing potential (including breastfeeding partner) must agree to use contraception throughout the treatment period and for 6 months after the last dose of study treatment administration.

Male subject must not donate sperm during the treatment period and for 6 months after the last dose of study treatment administration.

Male subject with pregnant partner(s) must agree to remain abstinent or use a condom for the duration of the pregnancy throughout the study period and for 6 months after the last dose of study treatment administration.

Subject agrees not to participate in another interventional study while receiving study treatment in the present study.

Disease Specific Inclusion Criteria:

Cohort 1: HR+/HER2− Breast Cancer

Subject has histologically or cytologically-confirmed HR+/HER2− breast cancers; defined as ER positive and/or progesterone receptor (PR) positive, and HER2 negative as per American Society of Clinical Oncology/College of American Pathologists (ASCO/CAP) guidelines based on the most recently analyzed tissue.

Subject has locally advanced or metastatic disease.

Subject must have received ≥1 line of endocrine therapy and a cyclin-dependent kinase (CDK) 4/6 inhibitor in the metastatic or locally advanced setting.

Subject must have received prior treatment with a taxane or anthracycline in any setting. Subject with a deleterious germline mutation in breast cancer susceptibility gene (BRCA)1 or 2 must have been treated with a poly ADP ribose polymerase (PARP) inhibitor.

Cohort 2: Triple Negative Breast Cancer

Subject has histologically or cytologically-confirmed TNBC; defined as unequivocal TNBC histology (ER-negative/PR-negative/HER2−negative) as per ASCO/CAP guidelines based on the most recently analyzed tissue.

Subject has locally advanced or metastatic disease.

Subject must have had ≥2 lines of systemic therapy, including a taxane in any setting. Subject with a deleterious germline mutation in BRCA1 or 2 must have been treated with a PARP inhibitor.

Cohort 3: Squamous Non-Small Cell Lung Cancer

Subject has histologically- or cytologically-confirmed squamous NSCLC.

Subject has locally advanced or metastatic disease.

Subject must have progressed or relapsed following platinum-based therapy; platinum therapy administered in the adjuvant setting counts as a regimen if relapse occurred within 12 months after completion.

Subject must have received prior therapy with an anti-programmed cell death protein-1 (PD-1) or anti-programmed cell death-ligand 1 (PD-L1) if eligible based on subject's tumor PD-1 or PD-L1 expression and local treatment guidelines.

Cohort 4: Non-Squamous Non-Small Cell Lung Cancer

Subject has histologically or cytologically-confirmed non-squamous NSCLC (epidermal growth factor receptor [EGFR] wild type and anaplastic lymphoma kinase [ALK] wild type by local laboratory standards).

Subject has locally advanced or metastatic disease.

Subject must have progressed or relapsed following platinum-based therapy in the metastatic or locally advanced setting; platinum therapy administered in the adjuvant setting counts as a regimen if relapse occurred within 12 months after completion.

Subject must have received an anti-PD-1 or anti-PD-L1 if eligible based on subject's tumor PD-1 or PD-L1 expression and local treatment guidelines.

Cohort 5: Head and Neck Cancer

Subject has histologically- or cytologically-confirmed head and neck cancer.

Subject has locally advanced or metastatic disease.

Subject must have progressed or relapsed following platinum containing regimen in the metastatic or locally advanced setting. Platinum regimens administered as part of multimodal therapy in the curative setting do not count as a prior regimen unless the subject relapsed or progressed within 6 months after completion.

Subject must have received an anti-PD-1 or anti-PD-L1 if eligible based on subject's tumor PD-1 or PD-L1 expression and local treatment guidelines.

Cohort 6: Gastric or Esophageal Cancer

Subject has histologically- or cytologically-confirmed gastric or esophageal cancer.

Subject has locally advanced or metastatic disease.

Subject must have progressed or relapsed following chemotherapy regimens that included a fluoropyrimidine and a platinum for metastatic disease or advanced disease. Neoadjuvant or adjuvant regimens do not count as a prior regimen unless the subject relapsed or progressed within 6 months after completion. Subject with HER2 positive cancer must have received HER2 directed therapy.

6.4.5.2. Exclusion Criteria

Subject is excluded from participation in the study if any of the following apply:

For all subjects in cohorts 1 to 6:

Subject has received ≥3 lines of prior chemotherapy-inclusive regimens for metastatic disease (no limit on non-chemotherapy regimens).

Subject has preexisting sensory or motor neuropathy Grade ≥2.

Subject has active central nervous system (CNS) metastases. Subjects with treated CNS metastases are permitted on study if all the following are true:

CNS metastases have been clinically stable for ≥6 weeks prior to screening

If requiring steroid treatment for CNS metastases, the subject is on a stable dose ≤20 mg/day of prednisone or equivalent for ≥2 weeks

Baseline imaging scans show no evidence of new or enlarged brain metastasis

Subject does not have leptomeningeal disease

Subject has ongoing clinically significant toxicity (Grade 2 or higher with the exception of alopecia) associated with prior treatment (including systemic therapy, radiotherapy or surgery).

Subject with ≤Grade 2 immunotherapy-related hypothyroidism or panhypopituitarism may be enrolled when well-maintained/controlled on a stable dose of hormone replacement therapy (if indicated). Subjects with ongoing ≥Grade 3 immunotherapy-related hypothyroidism or panhypopituitarism are excluded. Subjects with ongoing immunotherapy-related colitis, uveitis, myocarditis or pneumonitis, or subjects with other immunotherapy-related AEs requiring high doses of steroids (>20 mg/day of prednisone or equivalent), are excluded.

Subject has a history of uncontrolled diabetes mellitus within 3 months of the first dose of study treatment. Uncontrolled diabetes is defined as hemoglobin A1c (HbA1c)≥8% or HbA1c between 7 and <8% with associated diabetes symptoms (polyuria or polydipsia) that are not otherwise explained.

Subject has prior treatment with enfortumab vedotin or other monomethyl auristatin E (MMAE) based ADCs.

Subject has a second malignancy diagnosed within 3 years before first dose of study drug, or any evidence of residual disease from a previously diagnosed malignancy. Subjects with non-melanoma skin cancer, localized prostate cancer treated with curative intent with no evidence of progression, low-risk or very low-risk (per standard guidelines) localized prostate cancer under active surveillance/watchful waiting without intent to treat, or carcinoma in situ of any type (if complete resection was performed) are allowed.

Subject is currently receiving systemic antimicrobial treatment for viral, bacterial, or fungal infection at the time of first dose of study treatment. Routine antimicrobial prophylaxis is permitted.

Subject has known active hepatitis B (e.g., hepatitis B surface antigen [HBsAg] reactive) or active hepatitis C (e.g., hepatitis C virus [HCV] RNA [qualitative] is detected).

Subject has known history of human immunodeficiency virus (HIV) infection (HIV 1 or 2).

Subject has documented history of a cerebral vascular event (stroke or transient ischemic attack), unstable angina, myocardial infarction or cardiac symptoms (including congestive heart failure) consistent with New York Heart Association Class III-IV within 6 months prior to the first dose of study drug.

Subject has major surgery within 4 weeks prior to first dose of study drug.

Subject had radiotherapy, chemotherapy, biologics, investigational agents, and/or antitumor treatment with immunotherapy that is not completed 2 weeks prior to first dose of study drug.

Subject has known hypersensitivity to enfortumab vedotin or to any excipient contained in the drug formulation of enfortumab vedotin (including histidine, trehalose dihydrate and polysorbate 20) OR subject has known hypersensitivity to biopharmaceuticals produced in CHO cells.

Subject has known active keratitis or corneal ulcerations. Subject with superficial punctate keratitis is allowed if the disorder is being adequately treated in the opinion of the investigator.

Subject has any condition, which, in the investigator's opinion, makes the subject unsuitable for study participation.

6.4.6. Therapeutic Agent

6.4.6.1. Therapeutic Agent Administered

The therapeutic agent, enfortumab vedotin (ASG-22CE), is a sterile, preservative-free, white to off-white lyophilized powder to be reconstituted for intravenous administration. The therapeutic agent is supplied in single-use glass vials containing 20 mg or 30 mg enfortumab vedotin in each vial. The therapeutic agent is stored at 2° C. to 8° C.

6.4.6.1.1. Dosing and Administration of

THERAPEUTIC AGENT

Enfortumab vedotin at a dose of 1.25 mg/kg is administered as an intravenous infusion over approximately 30 minutes on days 1, 8, and 15 of every 28-day cycle. In the absence of infusion-related reactions (IRRs), the infusion rate for all subjects is calculated in order to achieve an approximate 30-minute infusion period. Enfortumab vedotin must not be administered as an intravenous push or bolus. Enfortumab vedotin should not be mixed with other medications. At least 7 days must elapse between doses of enfortumab vedotin.

Subject weight must be measured during all relevant assessment time points as described in the Schedule of Assessments. Weight-based dosing is calculated using the subject's actual body weight. An exception to weight-based dosing is made for subjects weighing greater than 100 kg; doses are based on 100 kg for these individuals. The maximum dose permitted on this study is 125 mg.

Subjects are observed during enfortumab vedotin administration and for at least 60 minutes following the infusion for the first 3 cycles. All supportive measures consistent with optimal subject care are given throughout the study according to institutional standards.

The injection site is monitored closely for redness, swelling, pain, and infection during and at any time after administration. Subjects are advised to report redness or discomfort promptly at the time of administration or after infusion.

6.4.6.2. Randomization and Blinding

This is an open-label study. Subject enrollment and dispensation of enfortumab vedotin are performed via the interactive response technology (IRT) system. Prior to initiation of study treatment, study site personnel obtains the subject number and medication assignment from the IRT system. Specific IRT procedures are known to a person skilled in the art.

6.4.6.3. Dose Modification

Dose reduction to 1, 0.75 or 0.5 mg/kg is allowed depending on the type and severity of toxicity. Subjects requiring a dose reduction may be re-escalated by 1 dose level (i.e., subjects reduced to 0.75 mg/kg may only be re-escalated to 1 mg/kg) provided the toxicity does not require study drug discontinuation and has returned to baseline or ≤Grade 1. If the toxicity recurs, re-escalation is not permitted. Subjects with ≥Grade 2 corneal AEs are not permitted to dose re-escalate. Dose modification recommendations for enfortumab vedotin associated toxicity are presented in Table 8 and Table 9

Dose interruptions for other enfortumab vedotin associated toxicity is permitted at the discretion of the site investigator. Dose interruptions may last up to 8 weeks (2 cycles). Dose interruptions for subjects who are deriving clinical benefit from treatment may be extended beyond 8 weeks, if the subject's toxicity does not otherwise require permanent discontinuation. During dose interruptions, the schedule for response assessments is not adjusted.

TABLE 8
Recommended Dose Modifications for Enfortumab
Vedotin Associated Hematologic Toxicity*

| Grade 1 | Grade 2 | Grade 3 | Grade 4 |
|---|---|---|---|
| Continue at | Continue at same dose | Withhold dose until | Withhold dose until toxicity |
| same dose | level. | toxicity is ≤Grade 1 or | is ≤Grade 1 or has returned to |
| level. | For Grade 2 | has returned to baseline, | baseline, then reduce dose by |
| | thrombocytopenia | then resume treatment at | 1 dose level and resume treatment |
| | withhold dose until | the same dose level or | or discontinue at the discretion of |
| | toxicity is ≤Grade 1 | consider dose reduction | the investigator. |
| | or has returned to | by 1 dose level. | Transfusions or growth factors |
| | baseline, then resume | Transfusions or growth | may be used as indicated per |
| | treatment at the same | factors may be used as | institutional guidelines. |
| | dose level. | indicated per institutional | For anemia, treatment |
| | | guidelines. | discontinuation should be strongly |
| | | | considered. |

*Hematologic toxicity refers to anemia, thrombocytopenia, neutropenia and febrile neutropenia.

TABLE 9
Recommended Dose Modifications for Enfortumab Vedotin Associated Non-hematologic Toxicity

| Grade 1 | Grade 2 | Grade 3 | Grade 4 |
|---|---|---|---|
| Continue at same | Continue at same dose level, | Withhold dose until toxicity | For Grade 4 AEs, |
| dose level. | except in the event of | is ≤Grade 1 or has returned to | discontinue |
| If ocular symptoms | Grade 2 neuropathy or | baseline, then resume treatment | treatment.** |
| and/or changes | corneal AEs. | at the same dose level or consider | Grade 4 vomiting |
| in vision are | For Grade 2 neuropathy or | dose reduction by 1 dose level.** | and/or diarrhea |
| identified, the | corneal AEs, withhold dose | For Grade 3 neuropathy or | that improves |
| subject should | until toxicity is ≤Grade 1 or | corneal AEs, discontinue | to ≤Grade 2 within |
| be evaluated | has returned to baseline, and | treatment. | 72 hours with |
| with an | then resume treatment at the | For Grade 3 | supportive |
| ophthalmologic | same dose level. For the | hyperglycemia/elevated blood | management does |
| exam.* | second occurrence of | glucose, withhold study | not require |
| | Grade 2 neuropathy or | treatment. Resume treatment | discontinuation. |
| | corneal AE's withhold dose | once hyperglycemia/elevated | |
| | until toxicity is ≤Grade 1, | blood glucose has improved | |
| | and then reduce the dose by | to ≤Grade 2 and subject is | |
| | 1 dose level and resume | clinically and metabolically | |
| | treatment. | stable. | |
| | If ocular symptoms and/or | If ocular symptoms and/or | |
| | changes in vision are | changes in vision are identified, | |
| | identified, the subject should | the subject should be evaluated | |
| | be evaluated with an | with an ophthalmologic exam.* | |
| | ophthalmologic exam.* | | |

AE: adverse events
*Ophthalmologic exam should be performed by an ophthalmologist. In countries where optometrists can perform exams and prescribe medications, an optometrist may be used instead.
**Grade 3/4 electrolyte imbalances/laboratory abnormalities that are not associated with clinical sequelae and/or are corrected with supplementation/appropriate management within 72 hours of their onset do not require discontinuation (e.g., Grade 4 hyperuricemia). Grade 3 rash that is not limiting selfcare activities of daily living or associated with infection requiring systemic antibiotics does not require treatment interruption, provided symptoms are not severe and can be managed with supportive treatment.

6.4.7. Study Procedures and Assessments 6.4.7.1. Efficacy Assessments

Disease response and progression are evaluated using RECIST version 1.1 (see 6.4.8.3, RECIST version 1.1). Radiographic assessments are performed according to the Schedule of Assessments. Confirmed ORR, DOR, DCR, PFS, and OS are evaluated as per the Schedule of Assessments.

6.4.7.1.1. IMAGING FOR DISEASE ASSESSMENT (COMPUTED TOMOGRAPHY/MAGNETIC RESONANCE IMAGING/POSITRON EMISSION TOMOGRAPHY-COMPUTED TOMOGRAPHY SCAN)

Imaging/disease assessment is performed at screening/baseline and every 8 weeks (56 days ±7 days) from the first dose of study treatment throughout the study until the subject has radiologically-confirmed disease progression, initiates a new subsequent anticancer therapy, dies, withdraws consent, is lost to follow-up or the study closes, whichever occurs first. Baseline imaging performed prior to informed consent as standard of care may be used as long as it is performed within 28 days prior to the first dose of study treatment.

If disease response is assessed as CR or PR by investigator, a confirmatory imaging scan is required 4 weeks (28 days+7 days) after the first response. After 1 year on study treatment, the frequency of disease response assessments is reduced to every 12 weeks (84 days ±7 days).

Disease response and progression are assessed by an BICR. The treatment decisions are made based on site assessments of scans by RECIST Version 1.1.

Subjects who discontinue study treatment for reasons other than radiologically-confirmed disease progression by RECIST Version 1.1 continue to receive imaging scans every 8 weeks (56 days ±7 days) until subject has radiologically-confirmed disease progression, initiates a new anticancer therapy, dies, withdraws consent, lost to follow-up, or the study closes, whichever occurs first. Tumor imaging may also be performed whenever disease progression is suspected.

Brain scan and bone imaging are performed according to the Schedule of Assessments and are repeated at response assessment time points if metastases were identified at screening/baseline, or if metastasis is known or suspected, or as clinically indicated throughout the study.

A CT scan with contrast (chest, abdomen and brain) is the preferred modality for tumor assessment. Magnetic resonance imaging is acceptable if local standard practice or if CT scans are contraindicated in a subject (e.g., subject is allergic to contrast media). All other RECIST-approved scanning methods such as x-ray are optional. Additional instructions for imaging assessments can be found in the study procedures manual.

The assessment includes tumor measurements for target lesions, nontarget lesions and any new lesions. A response assessment is characterized for a given time point evaluation. At the end of study for each subject, the best overall response to the study regimen is derived. To ensure comparability, the screening and subsequent assessment of response are performed using identical techniques. The same individual assesses images for any 1 subject for the duration of the study if possible. It is recommended that repeat imaging be used for subjects with known brain metastases in order to follow the lesions throughout the study.

The site of disease progression including target, nontarget and/or new lesions are documented in the eCRF. Additional imaging may be performed at any time to confirm suspected progression of disease.

This study is analyzed based on the results of both independent and local (investigator) radiologic assessments. Copies of all imaging scans are collected throughout the study and analyzed according the standard known to a person of ordinary skill in the art.

6.4.7.1.2. Evaluation of Target Lesions

Complete Response (CR)

CR is defined as disappearance of all target and nontarget lesions. Any pathological lymph nodes (whether target or nontarget) must have reduction in short axis to <10 mm from baseline measurement.

Partial Response (PR)

PR is defined as at least a 30% decrease in the sum of diameters (longest for nonnodal lesions; short axis for nodal lesions) of target lesions taking as reference to the baseline sum diameters.

Stable Disease (SD)

SD is defined as neither sufficient decrease to qualify for PR nor sufficient increase to qualify for progressive disease taking as reference the smallest sum of diameters while on study drug.

Progressive Disease (PD)

PD is defined as at least a 20% increase in the sum of diameters (longest for nonnodal lesions; short axis for nodal lesions) of the target lesions, taking as reference the smallest sum on study (this includes the baseline sum if that is the smallest on study). In addition to the relative increase of 20%, the sum must also demonstrate an absolute increase of at least 5 mm. The appearance of 1 or more new lesions is also considered progression.

Evaluation of Non-target Lesions

To achieve unequivocal progression on the basis of nontarget lesions, there must be an overall level of substantial worsening in nontarget disease such that, even in presence of SD or PR of target lesions, the overall tumor burden has increased sufficiently to merit discontinuation of therapy. A modest increase in the size of 1 or more nontarget lesions is usually not sufficient to qualify for unequivocal progression.

Non-complete Response/Non-progressive disease

NonCR/NonPD of nontarget lesions is defined as persistence of 1 or more nontarget lesions.

Progressive Disease

PD of nontarget lesions is defined as unequivocal progression of existing nontarget lesions or the appearance of 1 or more new lesions.

6.4.7.1.3. Evaluation of Time Point Response

The response status at each time point for subjects with measurable disease at baseline is determined.

6.4.7.1.4. Survival Status

After radiologically-confirmed disease progression or initiation of subsequent anticancer therapy, subjects are contacted every 12 weeks in the long-term follow-up period for collection of survival status until death, withdrawal of consent, lost to follow-up, or study closure, whichever occurs first.

6.4.7.2. Additional Assessments

6.4.7.2.1. BIOMARKER(S)

The samples collected during the course of the study, for example at each time point of the study as described herein, can be analyzed for other biomarkers including DNA, RNA and protein, to investigate possible associations with mechanisms of resistance or sensitivity to study treatment, dynamic changes associated with study treatment (in terms of dose, safety, tolerability and efficacy, etc.) and method development or validation of diagnostic assays related to enfortumab vedotin.

Blood Sample for Biomarker Analysis

Blood samples for biomarker analysis are collected from all subjects at the time points indicated in the Sample Collection Schedule for the isolation of plasma, serum, and peripheral blood mononuclear cells (PBMCs). A subset of the plasma samples may be used for the next-generation sequencing (NGS) analysis of circulating free DNA (cfDNA). A subset of the serum samples may be used to characterize soluble 191P4D12 levels. The plasma, serum, and PBMC samples may undergo cytokine or immune-phenotyping analysis to identify markers of immune function and immune cell subsets. The blood samples can be used for additional analyses as described herein and known to a person of ordinary skill in the art.

Tumor Tissue Samples for Biomarker Analysis

Pretreatment tumor tissue sample (from primary or metastatic site) in the form of a formalin-fixed, paraffin-embedded tumor tissue block or unstained charged slides as indicated in the Schedule of Assessments is collected. Either archival or pretreatment fresh tumor tissue is acceptable. If an archival tumor tissue sample is not available, the subject can have a biopsy to obtain tumor tissue prior to study treatment. If freshly sectioned, unstained charged slides are collected, a minimum of 10 and up to 20 slides are needed from each subject for the planned biomarker studies.

If biopsy is performed as standard of care while on study treatment or during follow-up period, subject's tumor tissue samples are collected and assessed as described herein.

The tumor tissue samples can be analyzed for 191P4D12 and PD-L1 expression, markers of disease subtype and markers related to the tumor immune microenvironment. The tumor tissue sample can be used for additional analyses as described herein and known to a person of ordinary skill in the art.

6.4.7.2.2. Quality of Life and Patient Reported Outcome Assessment

Pain Assessment

The subject rates his/her pain on a 0 to 10 scale that best describes the pain at its worst in the last 24 hours as indicated in the Schedule of Assessments. See 6.4.8.6 Pain Assessment.

Euro Quality of Life-5 Dimensions

At day 1 of each cycle and end of treatment, subject completes the EQ-5D-5L questionnaires. The EQ-5D-5L is a standardized instrument developed by the EuroQOL Group for use as a generic, preference-based measure of health outcomes. It is applicable to a wide range of health conditions and treatments and provides a simple descriptive profile and a single index value for health status. The EQ-5D-5L is a 5-item self-reported measure of functioning and well-being, which assesses 5 dimensions of health, including mobility, selfcare, usual activities, pain/discomfort, and anxiety/depression. Each dimension comprises 5 levels (no problems, slight problems, moderate problems, severe problems, extreme problems). A unique EQ-5D-5L health state is defined by combining 1 level from each of the 5 dimensions. This questionnaire also records the respondent's self-rated health status on a vertical graduated (0 to 100) visual analogue scale. Responses to the 5 items also be converted to a weighted health state index (utility score) based on values derived from general population samples (Herdman, Metal., Qual Life Res 2011; 20(10):1727-36). See 6.4.8.5 EQ-5D-5L.

6.4.8. Patient Outcome and Analyses Thereof

6.4.8.1. ANALYSIS OF EFFICACY

Efficacy analysis is conducted on the FAS, RES, and RES-BICR. Tumor related analyses are summarized based on RECIST version 1.1. Efficacy endpoints related to tumor assessment are analyzed for both BICR and investigator assessment.

6.4.8.1.1. Analysis of Primary Endpoint

Primary Analysis

The primary efficacy endpoint is the confirmed ORR per BICR. Confirmed ORR is defined as the proportion of subjects whose BOR is a confirmed CR or PR according to RECIST version 1.1. The confirmed ORR for each cohort is calculated and its 95% confidence interval is constructed by the Clopper-Pearson method. The primary analysis set for confirmed ORR per BICR is RES-BICR. Additional analysis on FAS and RES may also be performed.

6.4.8.1.2. Analysis of Secondary Endpoints

Duration of Response

DOR is defined as the time from the date of first documented response (CR or PR that is subsequently confirmed) to the date of first documented PD per RECIST version 1.1 or death due to any cause, whichever occurs first. DOR is only calculated for subjects achieving a confirmed CR or PR. DOR is analyzed using Kaplan-Meier methodology and Kaplan-Meier plots are provided. The median DOR and its 2-sided 95% CI are calculated.

Disease Control Rate

DCR is defined as the proportion of subjects whose BOR is confirmed CR or PR or SD. DCR for each cohort is calculated and its 95% confidence interval is constructed by Clopper-Pearson method.

Progression-free Survival

PFS is defined as the time from start of study treatment to first documentation of PD per RECIST version 1.1 or death due to any cause, whichever comes first. PFS is analyzed using Kaplan-Meier methodology and Kaplan-Meier plots are provided. The median PFS and its 2-sided 95% CI are calculated.

Overall Survival

OS is defined as the time from start of study treatment to date of death due to any cause. OS is analyzed using Kaplan-Meier methodology and Kaplan-Meier plots are provided. The median OS and its 2-sided 95% CI are calculated.

6.4.8.1.3. Analysis of Other Efficacy Endpoints

Best Overall Response

BOR is determined based on all available tumor time point response data for the subject. Responses recorded after new anticancer therapy or progressive disease (PD), are excluded from BOR derivation. A frequency table of BOR is presented for each cohort and overall.

The BOR is derived according to below criteria per RECIST version 1.1:

If a subject has at least 2 CR and the first and the last CR dates are more than 28 days apart, then the BOR is defined as confirmed CR.

If a subject has PR and another CR/PR more than 28 days apart, then the BOR for this subject is confirmed PR.

For those subject who do not have confirmed CR or PR, if the subject has at least 1 tumor assessment record of CR/PR/SD which is at least 49 days after the date of the first dose, then BOR is defined as SD.

For subjects who do not have confirmed CR, confirmed PR or SD defined as above, but they have the last tumor assessment as PD, their BOR is PD.

Otherwise, BOR is defined as Not Evaluable (NE) or No Data (ND) for the subjects without any post-baseline tumor assessment data.

Sum of Diameters

Per RECIST version 1.1, tumor burden is measured by the sum of diameters (SOD) of all target lesions at each tumor assessment. The maximum percent reduction from baseline in SOD is calculated for each subject and presented graphically with a waterfall plot.

Time to Response

Time to response (TRR) is calculated as the time from the first dose of study drug to the first documentation of objective response (CR or PR that is subsequently confirmed). TTR is only calculated for subjects achieving a confirmed CR or PR. TTR is summarized by descriptive statistics.

6.4.8.2. Other Analyses

6.4.8.2.1. Analysis of Biomarker(s)

Associations between potential genomic and/or other biomarkers (including 191P4D12 expression), and clinical results (efficacy, safety or pharmacodynamics) may be performed on subjects who have the necessary baseline and on study measurements to provide interpretable results for specific parameters of interest. Biomarkers may be summarized graphically or descriptively as they relate to clinical measures, as applicable. Summary statistics may be tabulated. Additional post-hoc analyses, such as alternative modeling approaches, may be conducted. All analyses described in this section are based on availability of the data.

6.4.8.2.2. Analysis of Quality of Life and Patient Reported Outcome Parameters

Descriptive QOL and PRO analyses are performed. Completion rate for each questionnaire is summarized. 6.4.8.3. RECIST VERSION 1.1

TABLE 1
Time point response: patients with target (+/−non-target) disease.

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or | No | PR |
| | not all evaluated | | |
| SD | Non-PD or | No | SD |
| | not all evaluated | | |
| Not all | Non-PD | No | NE |
| evaluated | | | |
| PD | Any | Yes or No | PD |
| Any | PD | Yes or No | PD |
| Any | Any | Yes | PD |

CR = complete response, PR = partial response, SD = stable disease, PD = progressive disease, and NE = inevaluable.

TABLE 2
Time point response: patients with non-target disease only.

| Non-target lesions | New lesions | Overall response |
|---|---|---|
| CR | No | CR |
| Non-CR/non-PD | No | Non-CR/non-PD^{a} |
| Not all evaluated | No | NE |
| Unequivocal PD | Yes or No | PD |
| Any | Yes | PD |

CR = complete response, PD = progressive disease, and NE = inevaluable.
^{a}‘Non-CR/non-PD’ is preferred over ‘stable disease’ for non-target disease since SD is increasingly used as endpoint for assessment of efficacy in some trials so to assign this category when no lesions can be measured is not advised.

TABLE 3
Best overall response when confirmation of CR and PR required.

| Overall | Overall | |
|---|---|---|
| response | response | |
| First | Subsequent | |
| time point | time point | BEST overall response |
| CR | CR | CR |
| CR | PR | SD, PD or PR^{a} |
| CR | SD | SD provided minimum criteria for SD |
| | | duration met, otherwise, PD |
| CR | PD | SD provided minimum criteria for SD |
| | | duration met, otherwise, PD |
| CR | NE | SD provided minimum criteria for SD |
| | | duration met, otherwise NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise NE |
| NE | NE | NE |

CR = complete response, PR = partial response, SD = stable disease, PD = progressive disease, and NE = inevaluable.
^{a}If a CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes 'CR' may be claimed when subsequent scans suggest small lesions were likely still present and in fact the patient had PR, not CR at the first time point. Under these circumstances, the original CR should be changed to PR and the best response is PR.
Reproduced from: Eisenhauer E A, Therasse P, Bogaerts J, Schwartz L H. Sargent D, Ford R, et al. New response evaluation criteria in solid tumors: revised RECIST guideline (version 1.1). Eur J Cancer. 2009; 45: 228-47.

6.4.8.4. EASTERN COOPERATIVE ONCOLOGY GROUP PERFORMANCE STATUS

| GRADE | ECOG PERFORMANCE STATUS |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry |
| | out any work activities; up and about more than 50% of |
| | waking hours |
| 3 | Capable of only limited selfcare; confined to bed or chair |
| | more than 50% of waking hours |
| 4 | Completely disabled; cannot carry on any selfcare; totally |
| | confined to bed or chair |
| 5 | Dead |

ECOG: Eastern Cooperative Oncology Group
Reproduced from: Oken M M, Creech R H, Tormey D C, Horton J, Davis T E, McFadden E T, et al. Toxicity and response criteria of the Eastern Cooperative Oncology Group. Am J Clin Oncol. 1982; 5: 649-55.

6.4.8.5. Eq-5D-5L

6.4.8.6. Pain Assessment

### 6.4.8.7. List of Abbreviations and Definition for the Human Clinical Study

List of Abbreviations

| Abbreviations | Description of abbreviations |
|---|---|
| ADC | antibody-drug conjugate |
| AE | adverse event |
| ALK | anaplastic lymphoma kinase |
| ALP | alkaline phosphatase |
| ALT | alanine aminotransferase |
| AST | aspartate aminotransferase |
| AT | aminotransferase |
| ATA | antitherapeutic antibodies |
| BICR | Blinded independent central review |
| BOP2 | Bayesian optimal design for phase 2 |
| BOR | best overall response |
| CHO | Chinese Hamster Ovary |
| CNS | central nervous system |
| CR | complete response |
| CRF | case report form |
| CT | computed tomography |
| DCR | disease control rate |
| DOR | duration of response |
| ECD | extracellular domain |
| ECG | electrocardiogram |
| ECOG | Eastern Cooperative Oncology Group |
| EGFR | epidermal growth factor receptor |
| EOT | end of treatment |
| EQ-5D-5L | EuroQOL 5-dimensions |
| ER | estrogen receptor |
| FAS | full analysis set |
| FDA | Food and Drug Administration |
| GFR | glomerular filtration rate |
| GI | gastrointestinal |
| HCG | human chorionic gonadotropin |
| HIV | human immunodeficiency virus |
| HPV | human papillomavirus |
| ICF | informed consent form |
| IDMC | Independent Data Monitoring Committee |
| IEC | Independent Ethics Committee |
| IRB | Institutional Review Board |
| IRT | Interactive response technology |
| MMAE | monomethyl auristatin E |
| NGS | next-generation sequencing |
| NSCLC | non-small cell lung cancer |
| ORR | objective response rate |
| OS | overall survival |
| PARP | poly ADP ribose polymerase |
| PBMC | peripheral blood mononuclear cells |
| PD | progressive disease |
| PFS | progression-free survival |
| PK | pharmacokinetics |
| PR | partial response |
| PRO | patient report outcome |
| QOL | quality of life |
| RECIST | Response Evaluation Criteria in Solid Tumors |
| RES | response evaluable set |
| SD | stable disease |
| SOD | sum of diameters |
| TEAE | Treatment-emergent adverse event |
| TNBC | triple negative breast cancer |
| TTR | time to response |
| UC | urothelial cancer |
| ULN | upper limit of normal |
| WOCBP | women of childbearing potential |

6.4.8.8. DEFINITION OF TERMS USED IN THE HUMAN STUDY

| Terms | Definition of Terms |
|---|---|
| Baseline | Assessments of subjects as they enter a study before |
| | they receive any treatment. |
| Endpoint | Variable that pertains to the efficacy or safety |
| | evaluations of a study. |
| | Note: Not all endpoints are themselves assessments |
| | since certain endpoints might apply to populations or |
| | emerge from analysis of results. That is, endpoints |
| | might be facts about assessments (e.g., prolongation |
| | of survival). |
| Enroll | To register or enter a subject into a study after |
| | screening. |
| Intervention | The drug, device, therapy or process under investiga- |
| | tion in a study that is believed to have an effect on |
| | outcomes of interest in a study (e.g., health-related |
| | QOL, efficacy, safety and pharmacoeconomics). |
| Screening | A process of active consideration of potential subjects |
| | for enrollment in a study. |
| Screen | Potential subject who signed the ICF but did not meet |
| failure | 1 or more criteria required for participation in the |
| | study and was not enrolled. |
| Screening | Period of time before entering the investigational |
| period | period, usually from the time when a subject signs the |
| | consent form until just before the test product or |
| | comparative drug (sometimes without randomization) is |
| | given to a subject. The investigational period is the |
| | period of time where major interests of protocol |
| | objectives are observed, and where the test product or |
| | comparative drug (sometimes without randomization) is |
| | given to a subject and continues until the last assess- |
| | ment after completing administration of the test product |
| | or comparative drug. |
| Study period | Period of time from the first study site initiation date |
| | to the last study site completing the study. |

### SEQUENCE LISTING

The present specification is being filed with a computer readable form (CRF) copy of the Sequence Listing. The CRF entitled 14369-248-228_SEQ_LISTING.txt, which was created on Jul. 30, 2020, is 39,675 bytes in size, and is incorporated herein by reference in its entirety.

1. A method of preventing or treating cancer in a subject, comprising administering to the subject an effective amount of an antibody drug conjugate,
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and
wherein the subject has:
(a) hormone receptor positive and human epidermal growth factor receptor 2 negative (HR+/HER2−) breast cancer;
(b) ER negative, PR negative, and HER2 negative (ER—/PR—/HER2−) breast cancer (Triple Negative Breast Cancer - TNBC);
(c) squamous non-small cell lung cancer (NSCLC);
(d) non-squamous NSCLC;
(e) locally advanced or metastatic head and neck cancer; or
(f) gastric or esophageal cancer.

2. The method of claim 1, wherein the subject has hormone receptor positive and human epidermal growth factor receptor 2 negative (HR+/HER2−) breast cancer; and
wherein the HR+/HER2− breast cancer is estrogen receptor (ER) positive and/or progesterone receptor (PR) positive, and HER2 negative.

3. The method of claim 1, wherein the subject has locally advanced or metastatic cancer.

4. The method of claim 1, wherein the subject has hormone receptor positive and human epidermal growth factor receptor 2 negative (HR+/HER2−) breast cancer; and
wherein the subject has previously received at least one line of an endocrine therapy and a cyclin-dependent kinase (CDK) 4/6 inhibitor in metastatic or locally advanced setting.

5. The method of claim 1, wherein the subject has hormone receptor positive and human epidermal growth factor receptor 2 negative (HR+/HER2−) breast cancer; and
wherein the subject has previously received a treatment with a taxane or anthracycline.

6. The method of claim 1, wherein the subject has hormone receptor positive and human epidermal growth factor receptor 2 negative (HR+/HER2−) breast cancer or ER negative, PR negative, and HER2 negative (ER—/PR—/HER2−) breast cancer (Triple Negative Breast Cancer - TNBC); and
wherein the subject has a deleterious germline mutation in breast cancer susceptibility gene (BRCA)1 or BRCA2, and wherein the subject has previously been treated with a poly ADP ribose polymerase (PARP) inhibitor.

7. (canceled)

8. (canceled)

9. The method of claim 1, wherein the subject has ER negative, PR negative, and HER2 negative (ER—/PR—/HER2−) breast cancer (Triple Negative Breast Cancer - TNBC); and
wherein the subject has previously received at least two lines of systemic therapies.

10. The method of claim 9, wherein the subject has previously received a treatment with a taxane.

11-13. (canceled)

14. The method of claim 1, wherein the subject has squamous non-small cell lung cancer (NSCLC); and
wherein the subject has progressed or relapsed following a platinum-based therapy.

15. The method of claim 14, wherein the subject has progressed or relapsed within 12 months after a platinum-based therapy.

16. The method of claim 1, wherein the subject has squamous non-small cell lung cancer (NSCLC), non-squamous NSCLC, or locally advanced or metastatic head and neck cancer; and
wherein the subject has previously received a therapy with an inhibitor of programmed cell death protein-1 (PD-1) or an inhibitor of programmed cell death-ligand 1 (PD-L1).

17. (canceled)

18. The method of claim 1, wherein the subject has non-squamous NSCLC; and
wherein the subject has wild-type epidermal growth factor receptor (EGFR) and wild-type anaplastic lymphoma kinase (ALK).

19. (canceled)

20. The method of claim 1, wherein the subject has non-squamous NSCLC; and
wherein the subject has progressed or relapsed following a platinum-based therapy.

21. The method of claim 20, wherein the subject has progressed or relapsed within 12 months after a platinum-based therapy.

22. (canceled)

23. (canceled)

24. The method of claim 1, wherein the subject has locally advanced or metastatic head and neck cancer, and wherein the subject has progressed or relapsed following a platinum-based therapy.

25. The method of claim 24, wherein the subject has progressed or relapsed within 6 months after a platinum-based therapy.

26-28. (canceled)

29. The method of claim 1, wherein the subject has gastric or esophageal cancer; and
wherein the subject has progressed or relapsed following a platinum-based therapy and/or a chemotherapy that included a fluoropyrimidine.

30. The method of claim 29, wherein the subject has progressed or relapsed within 6 months after the platinum-based therapy or the chemotherapy that included a fluoropyrimidine.

31. The method of claim 1, wherein the gastric or esophageal cancer is HER2 positive cancer, and wherein the subject has previously received a HER2 directed therapy.

32. The method of claim 1, wherein:
(a) the antibody or antigen binding fragment thereof comprises CDR H1 comprising the amino acid sequence of SEQ ID NO:9, CDR H2 comprising the amino acid sequence of SEQ ID NO:10, CDR H3 comprising the amino acid sequence of SEQ ID NO:11; CDR L1 comprising the amino acid sequence of SEQ ID NO:12, CDR L2 comprising the amino acid sequence of SEQ ID NO:13, and CDR L3 comprising the amino acid sequence of SEQ ID NO:14;
(b) the antibody or antigen binding fragment thereof comprises CDR H1 comprising the amino acid sequence of SEQ ID NO:16, CDR H2 comprising the amino acid sequence of SEQ ID NO:17, CDR H3 comprising the amino acid sequence of SEQ ID NO:18; CDR L1 comprising the amino acid sequence of SEQ ID NO:19, CDR L2 comprising the amino acid sequence of SEQ ID NO:20, and CDR L3 comprising the amino acid sequence of SEQ ID NO:21;
(c) the antibody or antigen binding fragment thereof comprises a heavy chain variable region comprising the amino acid sequence of SEQ ID NO:22 and a light chain variable region comprising the amino acid sequence of SEQ ID NO:23; or
(d) the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

33. (canceled)

34. (canceled)

35. The method of claim 1, wherein:
(a) the antigen binding fragment is an Fab, F(ab’)₂, Fv or scFv fragment;
(b) the antibody is a fully human antibody; or
(c) the antibody or antigen binding fragment thereof is recombinantly produced.

36. (canceled)

37. (canceled)

38. The method of claim 1, wherein the antibody drug conjugate has the following structure: 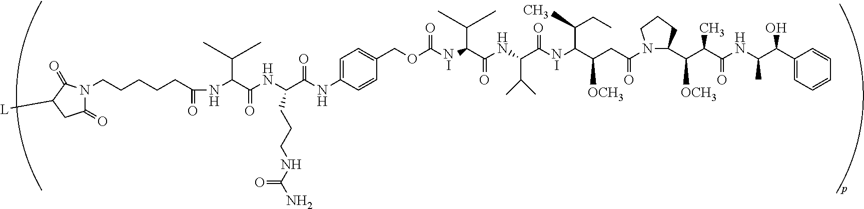
wherein L- represents the antibody or antigen binding fragment thereof and p is from 1 to 10, from 2 to 8, or from 3 to 5.

39. (canceled)

40. (canceled)

41. The method of claim 1, wherein the antibody or antigen binding fragment is linked to each unit of monomethyl auristatin E (MMAE) via a linker.

42. The method of claim 41, wherein:
(a) the linker is an enzyme-cleavable linker, and wherein the linker forms a bond with a sulfur atom of the antibody or antigen binding fragment thereof; or
(b) the linker has a formula of: —Aₐ—W_{w}—Y_{y}—; wherein —A— is a stretcher unit, a is 0 or 1; -W- is an amino acid unit, w is an integer ranging from 0 to 12; and -Y- is a spacer unit, y is 0, 1 or 2.

43. (canceled)

44. The method of claim 42, wherein:
(a) the stretcher unit has the structure of Formula (1) below; the amino acid unit is valine citrulline; and the spacer unit is a PAB group comprising the structure of Formula (2) below: 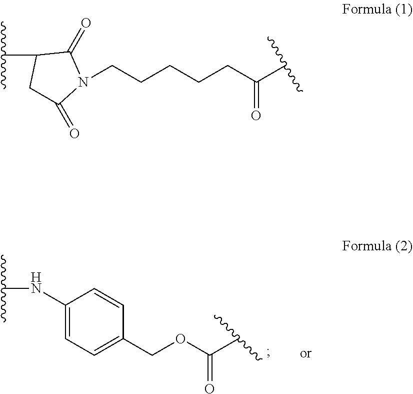 [SMILES: *C(=O)CCCCCN1C(=O)CC(*)C1=O ; *Nc1ccc(COC(*)=O)cc1]
or
(b) the stretcher unit forms a bond with a sulfur atom of the antibody or antigen binding fragment thereof and wherein the spacer unit is linked to MMAE via a carbamate group.

45. (canceled)

46. The method of claim 1, wherein:
(a) the antibody drug conjugate comprises from 1 to 10 units of MMAE per antibody or antigen binding fragment thereof;
(b) the antibody drug conjugate comprises from 2 to 8 units of MMAE per antibody or antigen binding fragment thereof or
(c) the antibody drug conjugate comprises from 3 to 5 units of MMAE per antibody or antigen binding fragment thereof.

47. (canceled)

48. (canceled)

49. The method of claim 1, wherein the antibody drug conjugate is administered at a dose of 1 to 10 mg/kg of the subject's body weight, 1 to 5 mg/kg of the subject's body weight, 1 to 2.5 mg/kg of the subject's body weight, 1 to 1.25 mg/kg of the subject's body weight, about 1 mg/kg of the subject's body weight, or about 1.25 mg/kg of the subject's body weight.

50. (canceled)

51. (canceled)

52. The method of claim 1, wherein:
(a) the antibody drug conjugate is administered by an intravenous (IV) injection or infusion;
(b) the antibody drug conjugate is administered by an intravenous (IV) injection or infusion over about 30 minutes twice every three-week cycle;
(c) the antibody drug conjugate is administered by an intravenous (IV) injection or infusion over about 30 minutes on Days 1 and 8 of every three-week cycle
(d) the antibody drug conjugate is administered by an intravenous (IV) injection or infusion over about 30 minutes three times every four-week cycle; or
(e) the antibody drug conjugate formulated in the pharmaceutical composition is administered by an intravenous (IV) injection or infusion over about 30 minutes on Days 1, 8 and 15 of every four-week cycle.

53-56. (canceled)

57. The method of claim 16, wherein the subject has previously received a therapy with an inhibitor of programmed cell death protein-1 (PD-1), wherein the inhibitor of PD-1 is nivolumab.

58. The method of claim 16, wherein the subject has previously received a therapy with an inhibitor of programmed cell death-ligand 1 (PD-L1), wherein the inhibitor of PD-L1 is selected from a group consisting of atezolizumab, avelumab, and durvalumab.


SEQUENCE LISTING (23 sequences)

SEQ ID NO: 1 - DNA, 3464 bp, Homo sapiens
  CDS (264)...(1796)
  misc_feature (1)...(3464): 191P4D12
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  misc_feature (1)...(510): 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  CDS (32)...(1432)
  misc_feature (1)...(1432): Ha22-2(2,4)6.1 heavy chain
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  misc_feature (1)...(466): Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  CDS (25)...(735)
  misc_feature (1)...(735): Ha22-2(2,4)6.1 light chain
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  misc_feature (1)...(236): Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  misc_feature (1)...(466): Ha22-2(2,4)6.1 heavy chain

SEQ ID NO: 8 - protein, 236 aa, Homo sapiens; identical to SEQ ID NO: 6
  misc_feature (1)...(236): Ha22-2(2,4)6.1 light chain

SEQ ID NO: 9 - protein, 5 aa, Homo sapiens
  CDR1 of heavy chain
SYNMN

SEQ ID NO: 10 - protein, 17 aa, Homo sapiens
  CDR2 of heavy chain
YISSSSSTIYYADSVKG

SEQ ID NO: 11 - protein, 8 aa, Homo sapiens
  CDR3 of heavy chain
AYYYGMDV

SEQ ID NO: 12 - protein, 11 aa, Homo sapiens
  CDR1 of light chain
RASQGISGWLA

SEQ ID NO: 13 - protein, 7 aa, Homo sapiens
  CDR2 of light chain
AASTLQS

SEQ ID NO: 14 - protein, 9 aa, Homo sapiens
  CDR3 of light chain
QQANSFPPT

SEQ ID NO: 15 - protein, 4 aa, Artificial Sequence
  linker
GFLG

SEQ ID NO: 16 - protein, 8 aa, Artificial Sequence
  VH CDR1 according to IMGT
GFTFSSYN

SEQ ID NO: 17 - protein, 8 aa, Artificial Sequence
  VH CDR2 according to IMGT
ISSSSSTI

SEQ ID NO: 18 - protein, 10 aa, Artificial Sequence
  VH CDR3 according to IMGT
ARAYYYGMDV

SEQ ID NO: 19 - protein, 6 aa, Artificial Sequence
  VL CDR1 according to IMGT
QGISGW

SEQ ID NO: 20 - protein, 3 aa, Artificial Sequence
  VL CDR2 according to IMGT
AAS

SEQ ID NO: 21 - protein, 9 aa, Artificial Sequence
  VL CDR3 according to IMGT
QQANSFPPT

SEQ ID NO: 22 - protein, 117 aa, Artificial Sequence
  heavy chain variable region (VH), the 20th amino acid (glutamic acid) to the 136th amino acid (serine) of SEQ ID NO7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, Artificial Sequence
  light chain variable region (VL), the 23rd amino acid (aspartic acid) to the 130th amino acid (arginine) of SEQ ID NO8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR